(19) United States
(12) Patent Application Publication
(10) Pub. No.: US 2014/0012251 A1
(43) Pub. Date: Jan. 9, 2014

(54) ABLATION DEVICES AND METHODS

(71) Applicant: Tidal Wave Technology, Inc., Memphis, TN (US)

(72) Inventors: Stevan Irwin Himmelstein, Memphis, TN (US); Michael C. Sherman, Memphis, TN (US); Roger A. Stern, Cupertino, CA (US); Jerome Jackson, Los Altos, CA (US)

(73) Assignee: Tidal Wave Technology, Inc., Memphis, TN (US)

(21) Appl. No.: 14/020,275

(22) Filed: Sep. 6, 2013

Related U.S. Application Data
(63) Continuation-in-part of application No. PCT/US2012/027849, filed on Mar. 6, 2012.
Application No. PCT/US2012/027849 is a continuation-in-part of application No. PCT/US2012/031582, filed on Mar. 30, 2012.
Application No. PCT/US2012/031582 is a continuation-in-part of application No. PCT/US2012/042664, filed on Jun. 15, 2012.
(60) Provisional application No. 61/450,016, filed on Mar. 7, 2011.
(60) Provisional application No. 61/470,383, filed on Mar. 31, 2011.
(60) Provisional application No. 61/497,366, filed on Jun. 15, 2011.
(60) Provisional application No. 61/793,024, filed on Mar. 15, 2013.

(51) Int. Cl. A61B 18/18

(52) U.S. Cl. CPC A61B 18/18
USPC 606/33

(57) ABSTRACT

An ablation device for denervation including a catheter delivery mechanism including an elongated tube with a distal end and a proximal end, the distal end being emplaceable within a body lumen at a target nerve region. A guide wire, at least one radiofrequency electrode, a plurality of positioning elements, and a plurality of pressing elements initially located within the tube. The electrode being deployable from the tube at the target nerve region and forming a ring-shaped structure adjacent the distal tube end. The positioning elements being deployable from the tube at the target nerve region from a position of the tube further distal than the electrode. The pressing elements being deployable from the tube more proximal than the electrode for use in pressing the deployed electrode against tissue to be ablated.

### CROSS REFERENCES TO RELATED APPLICATIONS

This application claims the benefit of priority of U.S. Provisional Patent Application No. 61/793,024, filed Mar. 15, 2013 and entitled “Ablation Catheter Devices and Methods;” and is a continuation-in-part of International Patent Application No. PCT/US2012/042664, filed Jun. 15, 2012 and entitled “Radiofrequency Ablation Catheter Device,” now published as WO 2012/174375 A1 and which claims the benefit of U.S. Provisional Patent Application No. 61/497,366 filed Jun. 15, 2011; and is a continuation-in-part of International Patent Application No. PCT/US2012/031582, filed Mar. 30, 2012 and entitled “Radio Frequency Ablation Catheter Device,” now published as WO 2012/135703 A2 and which claims the benefit of U.S. Provisional Patent Application No. 61/470,383 filed Mar. 31, 2011; and is a continuation-in-part of International Patent Application No. PCT/US2012/027849, filed Mar. 6, 2012 and entitled “Radiofrequency Ablation Catheter Device,” now published as WO 2012/122157 A1 and which claims the benefit of U.S. Provisional Patent Application No. 61/450,016 filed Mar. 7, 2011, all of which are expressly incorporated herein by reference in their entireties.

### FIELD

The present disclosure generally relates to a medical apparatus and method for treating neurovascular tissues through application of radiofrequency energy, and more particularly to an ablation apparatus for treating tissues in a patient and to delivering therapeutic radiofrequency energy through a catheter, stent or other similar device to a nerve site.

### BACKGROUND

Arteries are the tube-shaped blood vessels that carry blood away from the heart to the body's tissues and organs and are each made up of outer fibrous layer, smooth muscle layer, connecting tissue and the inner lining cells (endothelium). Certain arteries comprise complex structures that perform multiple functions. For example, the aorta is a complex structure that performs multiple functions. Arteries are often associated with a local network of nerves that are involved in many bodily functions including maintaining vascular tone throughout the entire body and each individual organ, sodium and water excretion or reabsorption, as in the kidney, and blood pressure control. The electrical activity to these nerves originates within the brain and the peripheral nervous system.

The kidneys have a dense afferent sensory and efferent sympathetic innervation and are thereby strategically positioned to be the origin as well as the target of sympathetic activation. Communication with integral structures in the central nervous system occurs via afferent sensory renal nerves. Renal afferent nerves project directly to a number of areas in the central nervous system, and indirectly to the anterior and posterior hypothalamus, contributing to arterial pressure regulation. Renal sensory afferent nerve activity directly influences sympathetic outflow to the kidneys and other highly innervated organs involved in cardiovascular control, such as the heart and peripheral blood vessels, by modulating posterior hypothalamic activity. These afferent and efferent nerves traverse via the aorta to their destination end-organ site.

Some studies suggest that conditions such as renal ischemia, hypoxia, and oxidative stress result in increased renal afferent activity. Stimulation of renal afferent nerves, which may be caused by metabolites, such as adenosine, that are formed during ischemia, uremic toxins, such as urea, or electrical impulses, increases reflex in sympathetic nerve activity and blood pressure.

An increase in renal sympathetic nerve activity increases renin secretion rate, decreases urinary sodium excretion by increasing renal tubular sodium reabsorption, and decreases renal blood flow and glomerular filtration rate. When nervous activity to the kidney is increased, sodium and water are reabsorbed, afferent and efferent arterioles constrict, renal function is reduced, and blood pressure rises.

Renin release may be inhibited with sympatholytic drugs, such as clonidine, moxonidine, and beta blockers. Angiotensin receptor blockers substantially improve blood pressure control and cardiovascular effects. However, these treatments have limited efficacy and adverse effects. In addition, many hypertensive patients present with resistant hypertension with uncontrolled blood pressure and end organ damage due to their hypertension.

Patients with renal failure and those undergoing hemodialysis treatment exhibit sustained activation of the sympathetic nervous system, which contributes to hypertension and increased cardiovascular morbidity and mortality. Signals arising in the failing kidneys seem to mediate sympathetic activation in chronic renal failure. Toxins circulating in the blood as a result of renal failure cause excitation of renal afferent nerves and may produce sustained activation of the sympathetic nervous system.

Abrogation of renal sensory afferent nerves and renal efferent nerves has been demonstrated to reduce both blood pressure and organ-specific damage caused by chronic sympathetic overactivity in various experimental models. Hence, functional denervation of the human kidney by targeting both efferent sympathetic nerves and afferent sensory nerves appears to be a valuable treatment strategy for hypertension and perhaps other clinical conditions characterized by increased overall nerve activity and particularly renal sympathetic nerve. Functional denervation in human beings may also reduce the potential of hypertension related end organ damage.

Destruction or reduction in size of cellular tissues in situ has been used in the treatment of many diseases and medical conditions, both alone and as an adjunct to surgical removal procedures. This procedure is often less traumatic than surgical procedures and may be the only alternative where other procedures are unsafe or ineffective. This method, known as ablative treatment (or therapy), applies appropriate heat (or energy) to the tissues and causes them to shrink and tighten. Ablative treatment devices have the advantage of using a destructive energy that is rapidly dissipated and reduced to a nondestructive level by conduction and convection forces of circulating fluids and other natural body processes.

In many medical procedures, it is important to be able to ablate the undesirable tissue in a controlled and focused way without affecting the surrounding desirable tissue. Over the years, a large number of minimally invasive methods have been developed to selectively destroy specific areas of undesirable tissues as an alternative to resection surgery. There are a variety of techniques with specific advantages and disadvantages, which are indicated and contraindicated for various applications.

In one technique, elevated temperature (heat) is used to ablate tissue. When temperatures exceed 60° C., cell proteins rapidly denature and coagulate, resulting in a lesion. The lesion can be used to resect and remove the tissue or to simply destroy the tissue, leaving the ablated tissue in place. Heat ablation can also be performed at multiple locations to provide a series of ablations, thereby causing the target tissue to die and necrose. Subsequent to heating, the necrotic tissue is absorbed by the body or excreted.

Electrical currents may be used to create the heat for ablation of the tissue. Radiofrequency ablation (RF) is a high temperature, minimally invasive technique in which an active electrode is introduced in the target area, and a high frequency alternating current of up to 500 kHz, for instance, is used to heat the tissue to coagulation. Radiofrequency (RF) ablation devices work by sending current through the tissue, creating increased intracellular temperatures and localized interstitial heat.

RF treatment exposes a patient to minimal side effects and risks, and is generally performed after first locating the tissue sites for treatment. RF energy, when coupled with a temperature control mechanism, can be supplied precisely to the apparatus-to-tissues contact site to obtain the desired temperature for treating a tissue. By heating the tissue with RF power applied through one or more electrodes from a controlled radio-frequency (RF) instrument, the tissue is ablated.

The general theory behind and practice of RF heat lesion has been known for decades, and a wide range of RF generators and electrodes for accomplishing such practice exist. RF therapeutic protocol has been proven to be highly effective when used by electrophysiologists for the treatment of tachycardia, by neurosurgeons for the treatment of Parkinson's disease, and by neurosurgeons and anesthetists for other RF procedures such as Gasserian ganglionectomy for trigeminal neuralgia and percutaneous cervical cordotomy for intractable pains, as well as raziotomy for painful facets in the spine.

More recently denervation of the kidney has been studied due to its well-known, positive impact on hypertension (high blood pressure). It can be accomplished, for example, via the renal artery ostium of the aorta, namely the orifice of the branch off the aorta that opens into the renal artery. Ablation of nerve activity at the level of the renal artery ostium will not affect blood flow from the aorta into the renal artery, but can cause the desired effect of denervation of the kidney. This kind of treatment is still relatively new, including what may be the best or desired treatment areas, and how to deliver the RF energy to the target area, which may be the area circumferentially surrounding the renal artery ostium. While the use of a catheter to deploy energy may be known for renal denervation, providing optimal uniform treatment is always a goal.

### SUMMARY

In general, this disclosure provides methods and improved medical ablation devices for effectively ablating a nerve function of a subject or patient.

In a first configuration, the improved medical ablation device delivers radiofrequency energy to the walls of a body lumen, particularly the renal artery, using a nonconductive catheter including a wire frame or stent that is expanded by inflating a balloon.

The device comprises a wire frame or stent bearing one or more electrodes that are capable of conducting RF energy. The one or more electrodes are positioned in a helical arrangement about the wire frame, which is positioned about an expandable balloon contained within a catheter, e.g., at the end thereof. The device is advanced over a guidewire within a sheath to the relevant location, such as within the renal artery, and positioned within the inner circumference of the vessel, such as the renal artery ostium. The sheath is then withdrawn to expose the balloon and wire frame on the catheter, and the wire frame or stent is then expanded by inflating the balloon at the end of the catheter. The wire frame or stent structure comprises at least one electrode that comes in contact with the body tissue when the system is expanded by the balloon.

The wire frame or stent is movable between a non-deployed position and a deployed position. In the non-deployed position, the balloon and wire frame are unexpanded, i.e., collapsed. The unexpanded balloon and wire frame in their non-deployed positions at the end of a catheter may be encapsulated within a sheath and advanced longitudinally through the blood vessel into the desired position, at which point the sheath may be withdrawn, exposing the unexpanded balloon and wire frame or stent member. The balloon is then expanded, thereby also expanding the wire frame into the deployed position, wherein it conforms to the walls of the lumen, so as to thereby allow the electrodes that are positioned about the wire frame to contact the lumen wall. Heat is then generated to the electrodes by supplying a suitable RF energy source to the apparatus, and the ablation is performed for the ablation of nerve activity, such as nerve activity that leads specifically to the kidney.

The device may comprise one or more ablation elements arranged in a helical fashion along the length of the expandable wire frame or stent that is positioned around the balloon catheter. For example, two or more, e.g., four, ablation elements may be arranged in a helical fashion along the length of the expandable wire cage or stent that is positioned around the balloon catheter. As another example, one linear array element may be arranged in a helical fashion along the length of the expandable wire cage or stent that is positioned around the balloon catheter. As yet another example, two linear array elements separated from each other by a predetermined distance are arranged in a helical fashion along the length of the expandable wire cage or stent that is positioned around the balloon catheter.

Positioning the RF elements in this helical fashion about the expandable wire cage or stent that is positioned around the catheter balloon allows the electrodes to be spaced along the surface of the renal artery, thereby ensuring improved delivery of the RF energy to the designated location within the renal arterial wall. By including multiple RF elements in a single catheter system, more complete nerve ablation is ensured.

Furthermore, a mechanism is provided in the catheter design for positioning and securing the catheter at the desired position within the vessel.

In one example, the device is a nonconductive flexible catheter for introduction into the lumen of a blood vessel, wherein the catheter has, near its remote end, an inflatable balloon that is connected to a balloon inflation and deflation source. A conductive wire is formed into a frame or stent and is situated in a collapsed position around the balloon when the balloon is in its deflated, non-deployed position. The wire frame may be made of a memory material such that the wire frame is in a collapsed state when the balloon is not inflated but assumes a generally cylindrical or helical shape when the balloon is advanced out of the catheter through a port and inflated. Alternatively, the wire frame may comprise interlocking or interwoven strands that are loosely interlocked or interwoven when the balloon is not inflated such that the wire frame is in a collapsed state and that, when the balloon is advanced out of the sheath and inflated, become more tightly interlocked or interwoven such that the wire frame assumes a generally cylindrical or helical shape and conforms to the walls of the lumen when the wire frame is in its deployed position.

Also included in this first configuration design is a mechanism to monitor catheter temperature during ablation, and a means to measure renal nerve afferent and effenert nerve activity prior-to and following RF nerve ablation. By measuring renal nerve activity post procedure, a degree of certainty is provided that proper nerve ablation has been accomplished. Renal nerve activity may be measured through the same electrode mechanism as that required for energy delivery.

In a second configuration, the improved medical ablation device delivers radiofrequency energy to the inner layer of a body lumen, particularly the aorta, specifically to the renal artery ostium of the aorta, using a nonconductive catheter also including a wire frame or stent, but with a different configuration of electrodes in comparison to the first configuration.

The device of this design comprises a wire frame or stent, e.g., cylindrically shaped, bearing one or more electrodes that are capable of conducting RF energy and that comes in contact with the body tissue. The one or more electrodes may have a circular configuration at one side of the wire frame. If more than one electrode is used, then the circular electrodes may be positioned concentrically. The wire frame is contacted against the inner surface of the aorta at the renal artery ostium, such that the circular electrodes ablate the nerve activity circumferentially around the renal artery ostium.

The wire frame or stent is movable between a non-deployed position and a deployed position. In the non-deployed position, the wire frame is unexpanded, i.e., collapsed. The collapsed wire frame in its non-deployed position at the end of a catheter may be encapsulated within a sheath. The device is advanced longitudinally through the blood vessel, e.g., over a guide wire, to the relevant location within the body lumen, such as within the aorta, and into the desired position within the inner circumference of the vessel, such as at the renal artery ostium of the aorta.

The sheath is then withdrawn, exposing the wire frame or stent member and allowing the wire frame to be expanded into the deployed position, wherein it conforms to the walls of the lumen, so as to thereby allow the electrodes that are positioned about the wire frame to contact the lumen wall. Heat is then generated to the electrodes by supplying a suitable RF energy source to the apparatus, and the ablation is performed for the ablation of nerve activity, such as nerve activity that leads specifically to the kidney.

The wire frame may be formed from (among other things) a material with a shape memory. The natural shape of the wire frame is in an expanded, generally cylindrical configuration, and the wire frame is positioned within the sheath in a collapsed configuration. When the sheath is withdrawn, the constraint on the wire frame keeping it in its collapsed configuration is released, allowing the wire frame to spontaneously expand to its remembered expanded configuration, in which it contacts the wall of the aorta.

Positioning the circularly-configured RF elements such that they are situated circumferentially around the opening to the renal artery ensures improved delivery of the RF energy to the designated location at the level of the aortic wall. By including multiple RF elements in a single catheter system, more complete nerve ablation may ensue.

Furthermore, a mechanism is provided in the catheter design for positioning and securing the catheter at the desired location within the vessel, e.g., the aorta, such that the electrodes can operate at the precise location, namely around the renal artery ostium. This mechanism will properly center the circularly-configured RF electrodes circumferentially around the opening to the renal artery. If the device is not properly positioned, the electrodes can ablate tissue that is not intended to be harmed, causing irreversible damage to other aortic or arterial structures.

An example of the positioning mechanism is an imaging catheter that allows the user to properly center and position the RF electrodes circumferentially around the opening to the renal artery. The imaging catheter allows the user to view exactly where the renal artery ostium is located. The distal end of the imaging catheter extends from the proximal direction into the wire frame and passes out through the hole of the circularly-configured RF electrodes. The circularly-configured RF electrodes can be positioned at the renal artery ostium by inserting the distal end of the imaging catheter at least partially into the entrance of the renal artery, to allow the device to hold its position within the aorta relative to the renal artery. When the device is so positioned, the wire frame can be expanded to the inner surface of the aorta, allowing the RF electrodes to be centered about the renal artery ostium while they perform their ablative function. Additionally, a balloon can be placed through the imaging catheter into the proximal segment of the renal artery for improved positioning and stabilization of the aortic device as discussed below.

The sheath that envelopes the device may have a longitudinal cut out to allow the imaging catheter/positioning device to protrude out of the wire frame and into the renal artery to position the device at the renal artery ostium, even while the wire frame is still in its collapsed, non-deployed configuration within the sheath and even while the sheath has not yet been withdrawn from over the wire frame. Once the device has been properly positioned, e.g., by insertion of the distal end of the imaging catheter at least partially into the entrance of the renal artery, the sheath is withdrawn and the wire frame is expanded. When the device has been properly positioned, expansion of the frame will result in its outer surface resting against the inside surface edges of the aorta, allowing the RF electrodes to be positioned against the renal artery ostium.

As another example, the positioning mechanism may comprise a balloon catheter with an inflatable balloon at its distal end that projects through the imaging catheter and into the entrance to the renal artery. This balloon catheter passes through the imaging catheter and the wire frame from the distal direction and passes through the hole of the circularly-configured RF electrodes, and is inserted at least partially into the entrance of the renal artery. The catheter sheath is then withdrawn and the balloon is then inflated, to allow the device to hold its position within the aorta relative to the renal artery. When the device is so positioned by virtue of the inflatable balloon, the device sheath is retracted so that the wire frame can be expanded to the inner surface of the aorta, allowing the RF electrodes to be positioned against the renal artery ostium so that they may perform their ablative function.

Also included in this second configuration design is a means to measure renal nerve afferent and efferent nerve activity prior-to and following RF nerve ablation. By measuring renal nerve activity post procedure, a degree of certainty is provided that proper nerve ablation has been accomplished. Renal nerve activity may be measured through the same electrode mechanism as that required for energy delivery at the level of the renal artery ostium, but also along the renal artery positioning balloon.

In a third configuration, the improved medical ablation device delivers radiofrequency energy to the inner layer of a body lumen, particularly the aorta, specifically surrounding the renal artery ostium of the aorta, using a nonconductive balloon catheter.

The device comprises a balloon catheter, e.g., cylindrically shaped, that may be expanded at some portions along its length through inflation. For example, the balloon catheter may be a noncompliant catheter that generally does not expand but has one or more separate compliant portions overlying the noncompliant catheter, which compliant portions may be separately or individually expandable through inflation. As another example, the balloon catheter may be a noncompliant catheter that generally does not expand but has one or more different compliant sections along its length, with each section having a different level of compliancy, to allow certain portions thereof to be expanded through inflation more than other portions thereof. And as yet another example, the balloon catheter may be a noncompliant catheter that generally does not expand but has one or more different compliant sections along its length, with each section having a different levels of compliancy, to allow certain portions thereof to be expanded through inflation more than other portions thereof, and also has one or more separate compliant portions overlying the catheter, which overlying compliant portions may be separately or individually expandable through inflation.

The device is movable between a non-deployed position and a deployed position. In the non-deployed position, the balloon catheter is unexpanded. In its non-deployed position, the balloon catheter may be advanced longitudinally through the blood vessel, e.g., over a guide wire and through a tube-like guiding catheter, to the relevant location within the body lumen, such as within the aorta, and into the desired position within the inner circumference of the vessel, such as at the renal artery ostium of the aorta.

The device may bear one or more electrodes that are capable of conducting RF energy and that come in contact with the body tissue. For example, the one or more electrodes may be positioned in a circular configuration on a portion of the balloon catheter when the device is in its deployed position. If more than one electrode is used, then the circularly configured electrodes may be positioned such that, when the device is in a deployed position, the electrodes together have a circular configuration or are oriented concentrically. The electrodes may be contacted against the inner surface of the lumen, e.g., the aorta, for example, at the renal artery ostium, such that the electrodes ablate the nerve activity circumferentially around the renal artery ostium.

When the device is in its deployed position, the compliant segment of the balloon catheter, called the balloon segment, is expanded such that it has a disk-like configuration with a circular, somewhat planar surface that is oriented orthogonally to the direction of the guide wire and facing in a distal direction. The one or more electrodes having a circular configuration are situated on the balloon segment of the device when the device is in its deployed position, i.e., on the distally-facing surface of the expanded catheter segment. This distally-facing surface of the balloon segment can be pressed up against the renal artery ostium of the aorta, such that electrodes that are positioned in a circular configuration may be made to contact the renal artery ostium of the aorta.

Heat is then generated to the electrodes by supplying a suitable RF energy source to the apparatus, and the ablation is performed for the ablation of nerve activity, e.g., at the renal artery ostium, such as nerve activity that leads specifically to the kidney. Positioning the circularly-configured RF elements such that they are situated circumferentially around the opening to the renal artery ensures improved delivery of the RF energy to the designated location at the level of the aortic wall. By including multiple RF elements in a single catheter system, more complete nerve ablation may ensue.

A mechanism may also be provided in the device design for positioning and securing the device at the desired location within the vessel, e.g., the aorta, such that the electrodes can operate at the precise location, namely around the renal artery ostium.

For example, the positioning mechanism may comprise a guide wire and unexpanded section of the balloon catheter that is inserted at least partially into the entrance to the renal artery and remains there. If there is a guiding catheter overlying the expandable catheter, the guiding catheter is then withdrawn proximally, and the balloon catheter segment is then inflated. The sheath or a guiding catheter is then advanced distally such that its distal edge presses against the proximally-facing surface of the expanded catheter segment, thereby allowing the RF electrodes on the distally-facing surface of the expanded catheter segment to be positioned against the renal artery ostium so that they may perform their ablative function.

As another example, the positioning mechanism may comprise a separately compliant portion of the balloon catheter, namely a separately inflatable portion that is situated distally of the balloon segment that projects into the entrance to the renal artery, called the positioning segment. This positioning segment of the balloon catheter is inserted at least partially into the entrance of the renal artery and is then inflated, not to the extent of the balloon catheter segment but only approximately to the diameter of the renal artery, so as to prevent the balloon catheter from being moved distally or proximally relative to the renal artery, so as to allow the device to hold its position within the renal artery relative to the aorta. When the device is so positioned by virtue of the inflatable balloon in the positioning segment of the balloon catheter, the circular RF electrodes may be positioned against the renal artery ostium so that they may perform their ablative function. Before the positioning segment of the balloon catheter is expanded, the distal edge of the sheath or guiding catheter may press against the proximally-facing surface of the expanded catheter segment, thereby allowing the RF electrodes on the distally-facing surface of the expanded balloon catheter segment to be positioned against the renal artery ostium

As another example, the positioning mechanism may comprise an imaging catheter at the distal end of the balloon catheter that allows the user to properly center and position the balloon catheter within the renal artery. The imaging catheter allows the user to view exactly where the renal artery ostium is located.

Once the device has been properly positioned, e.g., by one of the positioning means described above, the balloon segment of the balloon catheter is expanded. When the expanded balloon segment of the balloon catheter has been properly positioned, the distally-facing surface of the expanded balloon segment of the balloon catheter rests against the inside surface edges of the aorta, allowing the RF electrodes to be positioned against the aortic wall surrounding the renal artery ostium.

Also included in this third configuration design is a means to measure renal nerve afferent and efferent nerve activity prior-to and following RF nerve ablation. By measuring renal nerve activity post procedure, a degree of certainty is provided that proper nerve ablation has been accomplished. Renal nerve activity may be measured through the same electrode mechanism as that required for energy delivery at the level of the renal artery ostium, but also along the renal artery positioning balloon.

In a fourth configuration, the improved medical ablation device delivers radiofrequency energy to the inner layer of a body lumen, particularly neurovascular tissue being targeted which may be wrapped around the outside of the aorta and the renal artery, using a nonconductive catheter including an elongated tube.

The ablation device includes a catheter delivery mechanism including an elongated tube with a distal end and a proximal end, the distal end being placed within a body lumen at a target neurovascular region. A guide wire is disposed within the elongated tube. At least one radiofrequency electrode is initially located within the tube. The electrode being deployable from the tube at the target neurovascular region, and when deployed the electrode forms a ring-shaped structure generally centered about the tube adjacent the distal tube end. A plurality of positioning elements are initially located within the tube. The positioning elements are deployable from the tube at the target neurovascular region from a position of the tube further distal than the electrode. Pressing elements, initially located within the tube, are also deployable from the tube more proximal than the electrode for use in pressing, or positioning, the deployed electrode against tissue to be ablated. The tissue directly in contact with the electrode may be cooled by the device, thereby enabling targeting of an ablation deeper in the tissue without ablating the tissue in direct contact with the electrode. This is a case where the nerves being targeted are actually wrapped around the outside of the aorta and the renal arteries.

An example of a method for performing ablation of a neurovascular structure at an artery ostium, as in denervation, includes providing a catheter delivery mechanism including an elongated tube with a distal end and a proximal end, the distal end being emplaceable within a body lumen at a target neurovascular region, and having a guide wire within the elongated tube. Inserting the catheter delivery mechanism with its distal end at a target neurovascular region using the guide wire. At least one radiofrequency electrode initially located within the tube is provided, the electrode when deployed forming a ring-shaped structure generally centered about the tube adjacent the distal tube end. A plurality of positioning elements initially located within the tube are provided, the positioning elements being deployable from the tube at the target neurovascular region from a position of the tube further distal than the electrode. The positioning elements are then deployed to optimally position the electrode. The electrode is deployed at the target neurovascular region. A plurality of pressing elements initially located within the tube are provided, the pressing elements being deployable from the tube more proximal than the electrode for use in pressing the deployed electrode against tissue to be ablated to bring the electrode in close contact with the tissue. The electrode is pressed against the target neurovascular region, with radiofrequency energy applied through the deployed electrode from the tube at the target nerve region in an amount to ablate the targeted nerve region.

Another example of a method for performing ablation of a renal nerve at the renal artery ostium includes providing a catheter delivery mechanism including an elongated tube with a distal end and a proximal end, the distal end being emplaceable within the body lumen at the renal artery ostium, and having a guide wire within the elongated tube for positioning the catheter delivery mechanism. The catheter delivery mechanism is inserted with its distal end at the renal ostium. At least one radiofrequency electrode initially located within the tube is provided, the electrode when deployed forms a ring-shaped structure generally centered about the tube adjacent the distal tube end. A plurality of positioning elements initially located within the tube are provided, the positioning elements being deployable from the tube in the renal artery at the ostium from a position of the tube further distal than the electrode. The positioning elements are deployed to optimally center the electrode. The electrode is deployed and a plurality of pressing elements initially located within the tube are provided, the pressing elements being deployable from the tube more proximal than the electrode for use in pressing the deployed electrode against ostium tissue. In one aspect, it is not the ostium but the tissue deep behind the ostium that is targeted to ablate. The electrode is then pressed against the ostial tissue, and radiofrequency energy is applied through the deployed electrode from the tube in a pre-specified amount to ablate the neurovascular tissue wrapped around a backside of the aorta and the renal artery.

In addition to the above noted functions, each of these configurations of the device may also comprise a mechanism for cooling the aortic wall and the ostium in order to limit potential damage to the endothelial surface of the aorta while ablative energy is effectively transmitted to the adventitial layer. This cooling mechanism is by means of coolant or chilled material circulated through a hollow tube of the electrode, thus providing protection to the aortic wall at the level of the energy delivery. By cooling the tissue directly near the electrode, a target region deeper in the tissue (for example, tissue deep behind the ostium) can be ablated without ablating the tissue in direct contact with the electrode. This allows the target nerve region, a region wrapped around the outside of the aorta and the renal arteries, to be ablated when the device is deployed within the aorta and renal arteries.

If the configuration includes an expandable balloon, cooling also protects the balloon from high temperatures that might otherwise damage the integrity of the balloon. An insulation pad may be situated between each RF electrode and the surface of the balloon for insulating the balloon from the high temperatures of the RF electrode. Such an insulation pad avoids potential damage to the catheter balloon while ablative energy is effectively transmitted to the vessel surface. Coolant or chilled material may also be used to inflate the balloon, either in conjunction with or as an alternative to circulating coolant or chilled material through a hollow tube of the electrode.

The present disclosure is also directed to a method for radio-frequency (RF) heat ablation of tissue through the use of one or more RF electrodes. The RF electrodes may be deployed from the distal end of a catheter. For example, the RF electrodes may be positioned in a helical arrangement around a wire frame or stent that is mounted about a balloon positioned at the distal end of a catheter, as arranged in the first configuration. As another example, circular shaped RF electrodes may be mounted on a side of an expandable portion (e.g., a cylindrically-shaped wire frame or stent that is mounted in a compressed configuration) at the distal end of a catheter within a sheath, as arranged in the second configuration. The catheter may be inserted into the body via a natural orifice, a stoma or a surgically created opening that is made for the purpose of inserting the catheter, and insertion of the catheter may be facilitated with the use of a guide wire or a generic support structure or visualization apparatus. The catheter is advanced through the body to the relevant location, such as in the aorta at the location of the ostium of the renal artery.

The device may be positioned at the renal artery ostium of the aorta by use of a positioning mechanism. A positioning member may assist the user in determining where the renal artery ostium is. For example, in configurations including a wire frame, an imaging catheter may extend out of the wire frame so as to assist the user in determining where the renal artery is. As another example, in configurations including a balloon catheter extending out of the wire frame, the balloon may be inflated and center the RF elements circumferentially around the ostium of the renal artery.

Once the catheter is at the relevant location, the RF electrodes may be positioned, such as against the inner surface of the renal artery or aorta. For example, ablation may be performed for the aortic nerve activity that leads specifically to the kidney. As another example, a portion of the catheter may be expanded (e.g., expanding the balloon and/or wire frame or stent), positioning the RF electrodes mounted thereon against the inner surface of the aorta, at the ostium of a target branch artery. As another example, expanding the catheter may center the RF elements within the vessel, providing selective ablation of renal nerve activity leading to the kidney. The electrodes may also be positioned about the opening of the renal artery so as to surround the renal artery ostium.

The RF energy is applied to the RF electrodes in order to effect changes in the target tissue. Heat is generated by supplying a suitable energy source to the apparatus, which is comprised of at least one electrode that is in contact with the body tissues. Additionally, coolant—either stagnant or circulating—may be employed to cool the inner surface of the vessel wall. This coolant function may provide a form of protection or insulation to the inner vessel wall surface during RF energy activation and heat transfer.

### BRIEF DESCRIPTION OF DRAWINGS

Embodiments of devices, systems, and methods are illustrated in the figures of the accompanying drawings which are meant to be exemplary and not limiting, in which like references are intended to refer to like or corresponding parts, and in which:

FIGS. 1 and 2 illustrate example devices of the first configuration, fordelivering radiofrequency energy to the walls of a body lumen.

FIG. 3 is a block diagram illustrating a process for ablation of nerve function.

FIG. 4 illustrates an example device of the second configuration, for delivering radiofrequency energy to the renal artery ostium.

FIG. 5 is a cross-sectional exploded perspective view of the example device in FIG. 4

FIGS. 6 and 7 are further cross-sectional views of the device in FIG. 4.

FIG. 8 is a perspective view illustrating a sheath for use with the example device in FIG. 4.

FIG. 9 illustrates a side view of an example device of the third configuration for delivering radiofrequency energy, as to the renal artery ostium.

FIG. 10 illustrates a side view of the device in FIG. 9 in a deployed position.

FIG. 11 is a front end view of the device in FIG. 9 in a deployed position.

FIG. 12 illustrates a side view of another example of a device of the third configuration for delivering radiofrequency energy.

FIG. 13 illustrates an example of an ablation device of the third configuration.

FIG. 14 illustrates a delivery catheter for the ablation device of FIG. 13.

FIG. 15 illustrates a side view of an electrode of an ablation device of FIG. 13 deployed from the delivery catheter of FIG. 14.

FIG. 16 illustrates positioning and pressing elements of an ablation device as deployed from the delivery catheter of FIG. 14.

FIG. 17 illustrates the positioning elements, pressing elements, and electrode as deployed from the delivery catheter of FIG. 14.

FIG. 18 illustrates another embodiment of an electrode of the third configuration of an ablation device.

FIG. 19 illustrates a schematic of an example system including the ablation device of FIG. 16.

### DETAILED DESCRIPTION OF EMBODIMENTS

Detailed embodiments of devices, systems, and methods are disclosed herein, however, it is to be understood that the disclosed embodiments are merely exemplary of the devices, systems, and methods, which may be embodied in various forms. Therefore, specific functional details disclosed herein are not to be interpreted as limiting, but merely as a basis for the claims and as a representative basis for teaching one skilled in the art to variously employ the present disclosure.

As used herein, “proximal” refers to a portion of an instrument closer to an operator, while “distal” refers to a portion of the instrument farther away from the operator.

The term “subject” or “patient” refers in an embodiment to a mammal including a human in need of therapy for, or susceptible to, a condition or its sequelae. The subject or patient may include dogs, cats, pigs, cows, sheep, goats, horses, rats, and mice and humans.

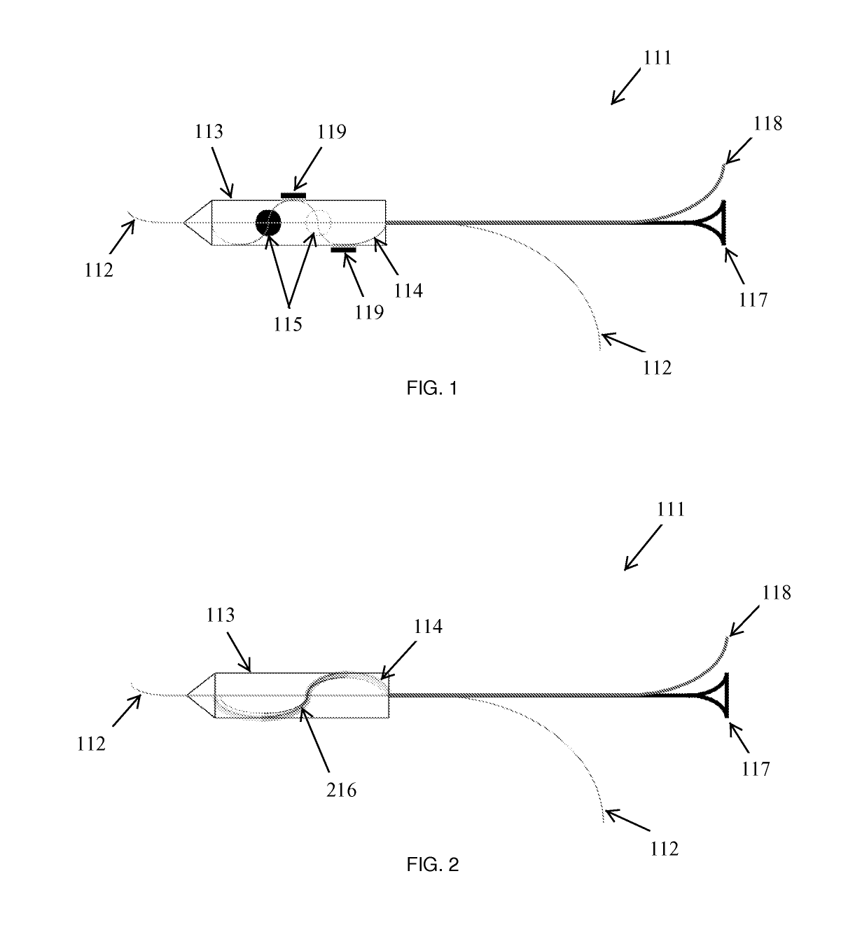

FIGS. 1 and 2 illustrate examples of devices based on the first configuration for delivering radiofrequency energy to the walls of a body lumen. Radiofrequency energy may be delivered, for example, to the walls of the renal artery or aorta using a nonconductive catheter 111.

The device includes a substantially tubular catheter 111, which may be a long, thin, tube-like device, having proximal and distal openings, preferably constructed from a nonconductive material. The catheter 111 may be any type of catheter, as are well known to those in the art, having a proximal end for manipulation by an operator and a distal end for operation within a patient. The distal end and proximal end preferably form one continuous piece.

As will be discussed in greater detail below, the catheter 111 is used as a delivery system for delivering a device containing radiofrequency electrodes 115,216 to the desired site for nerve ablation. As is known in the art, a guide wire 112 may first be inserted into the patient's vascular system and advanced to the desired location, and the catheter 111 is inserted into the patient and threaded over the guide wire 112 to the desired location.

The catheter 111 may include a positioning element. An example of a positioning element includes an inflatable balloon 113, of a type that is well known to those in the art, situated at the distal end of the catheter 111. The balloon 113 is pneumatically connected to a port at the proximal end of the catheter 111 and is thereby connected to a balloon inflation and deflation source for inflation and deflation of the balloon 113. The catheter 111 may be, among other things, a compliant balloon design that is advanced to the desired location within the patient's vascular system with, e.g., a rapid exchange (RX) or over-the-wire wire (OTW) delivery system. The uninflated balloon 113 may be situated within an outer catheter sleeve or sheath during insertion into the vessel, so as to prevent inadvertent inflation of the balloon 113 prior to placement at the desired site within the patient.

The catheter 111 may also include a thermal electric field delivery apparatus. For example, the thermal electric field delivery apparatus may comprise a wire frame 114 or stent positioned about the catheter's expandable balloon 113. The wire frame 114 may or may not be bonded to the balloon 113. The wire frame 114 may be conductive so as to be able to provide current to RF electrodes and temperature sensing functions.

The wire frame 114 is preferably situated in a collapsed position around the balloon 113 when the balloon 113 is in its deflated, non-deployed position. The wire frame 114 may be situated within an outer catheter sleeve during insertion into the vessel, so as to prevent inadvertent inflation of the balloon 113 and deployment of the wire frame 114.

The wire frame 114 may be made of a memory material such that the wire frame 114 is in a collapsed state when the balloon 113 is not inflated but assumes a generally cylindrical shape when the balloon 113 is advanced out of the catheter 111 through a port and inflated.

The wire frame 114 may also comprise interlocking or interwoven strands that are loosely interlocked or interwoven when the balloon 113 is not inflated such that the wire frame 114 is in a collapsed state. Then, when the balloon 113 is advanced out of the sheath and inflated, the interlocking or interwoven strands of the wire frame 114 or stent become more tightly interlocked or interwoven such that the wire frame 114 assumes a generally cylindrical or helical shape. The wire frame 114 conforms to the walls of the lumen when the wire frame 114 and balloon 113 are in their deployed position.

The wire frame 114 or stent is thus movable between a non-deployed position when the balloon 113 is unexpanded and a deployed position when the balloon 113 is expanded. It is also preferable that the wire frame 114 be collapsible, along with the balloon 113, back to its non-deployed position for retraction back into the catheter sheath along with the deflated balloon 113 after ablation is complete and when it is desired to withdraw the catheter 111 from the patient.

The wire frame 114 comprises at least one electrode 115,216 that is capable of conducting RF energy and that comes in contact with the body tissue when the system is expanded by the balloon 113. For example, as shown in FIG. 1, there are two or more helically placed electrodes 115. Preferably, there are four electrodes 115, although fewer or more than four electrodes 115 may also be used. By including multiple RF electrodes 115 in a single catheter system, more complete nerve ablation is ensured.

The individual electrodes 115 that are positioned along the wire frame 114 or stent are known as spot electrodes because they deliver thermal energy to a specific spot, as opposed to a larger area.

RF electrodes 115 are attached to the balloon 113 by means of the wire frame 114 that imparts support to the catheter 111 structure as well as providing a means to deliver RF energy and temperature and nerve activity sensing. The electrodes 115 contained in the set of electrodes may be evenly spaced around the circumference of the catheter balloon 113 and/or may be positioned in a helical fashion around the outside of the balloon 113. The purpose of positioning the electrodes 115 about the circumference of the catheter balloon 113 is so that the electrodes 115 would be situated along the circumference of the inside surface of the vessel, e.g., the renal artery, when the balloon 113 is expanded and the electrodes 115 are positioned against the vessel, for more effective ablation of, e.g., the renal nerve.

As illustrated in FIG. 2, the electrode is in the form of a ribbon-shaped electrode 216 that is positioned in a helical fashion around the outside of the balloon 113. If there is only one electrode 216 within the subject's body, known as a monopolar design, another electrode is positioned outside the subject's body, e.g., on the subject's skin.

However, the device may include more than one electrode 216. For example, the device may include two ribbon-shaped electrodes 216 that are positioned in a double-helical fashion around the outside of the balloon 113 (similar to a DNA strand). In such an embodiment where there are two electrodes 216 within the subject's body, known as a bipolar design, the two ribbon-shaped electrodes 216 are separated by a predetermined distance.

At the proximal end thereof, the catheter 111 includes at least two ports. A first port 117 is for connection to an air source for inflation and deflation of the balloon 113 and can be coupled to a pump or other apparatus to inflate or deflate the balloon 113 of the catheter 111. The balloon positioning device is pneumatically connected to the air source through the first port 117. This same port 117 may be used to circulate coolant to the inside of the balloon 113 for the purpose of cooling the balloon 113 during RF energy activation.

Another port 118 is for connection to a source of radiofrequency (RF) power and can be coupled to a source of Radiofrequency (RF) energy, such as RF in about the 300 kilohertz to 500 kilohertz range. The electrodes 115,216 are electrically coupled to the RF energy source through the second port 118. The catheter 111 may also be connected to a control unit for sensing and measurement of other factors, such as temperature, conductivity, pressure, impedance and other variables, such as nerve energy.

The RF electrodes 115,216 operate to provide radiofrequency energy for heating of the desired location during a nerve ablation procedure. Electrodes 115,216 may be constructed of any suitable conductive material, as is known in the art. Examples include stainless steel and platinum alloys.

RF electrodes 115,216 may operate in either bipolar or monopolar mode, as discussed above, with a ground pad electrode. In a monopolar mode of delivering RF energy, a single electrode 115,216 is used in combination with an indifferent electrode patch that is applied to the body to form the other electrical contact and complete an electrical circuit. Bipolar operation is possible when two or more electrodes 115,216 are used, either spot electrodes 115 or ribbon electrodes 216. Electrodes 115,216 can be attached to an electrode delivery member by the use of soldering methods which are well known to those skilled in the art.

The RF electrodes 115,216 also function to measure afferent and efferent nerve activity before and after vessel and nerve ablation.

Each electrode 115,216 can be disposed to treat tissue by delivering Radiofrequency (RF) energy. The radiofrequency energy delivered to the electrode 115,216 has a frequency of about 5 kilohertz (kHz) to about 1 GHz. In specific embodiments, the RF energy may have a frequency of about 10 kHz to about 1000 MHz; specifically about 10 kHz to about 10 MHz; more specifically about 50 kHz to about 1 MHz; even more specifically about 300 kHz to about 500 kHz.

The electrodes 115,216 may be operated separately or in combination with each other as sequences of electrodes disposed in arrays. Treatment can be directed at a single area or several different areas of a vessel by operation of selective electrodes. Different patterns of lesions, ablated, bulked, plumped, desiccated or necrotic regions can be created by selectively operating different electrodes 115,216. Production of different patterns of treatment makes it possible to remodel tissues and alter their overall geometry with respect to each other. In addition, varying the placement distance between bipolar electrodes will generate electrical fields allowing for temperature penetration of varying depths through the tissue.

An electrode selection and control switch may include an element that is disposed to select and activate individual electrodes 115,216.

RF power source may have multiple channels, delivering separately modulated power to each electrode 115,216 or array. This reduces preferential heating that occurs when more energy is delivered to a zone of greater conductivity and less heating occurs around electrodes 115,216 that are placed into less conductive tissue. If the level of tissue hydration or the blood infusion rate in the tissue is uniform, a single channel RF power source may be used to provide power for generation of lesions relatively uniform in size.

RF energy delivered through the electrodes 115,216 to the tissue causes heating of the tissue due to absorption of the RF energy by the tissue and ohmic heating due to electrical resistance of the tissue. This heating can cause injury to the affected cells and can be substantial enough to cause cell death, a phenomenon also known as cell necrosis. For ease of discussion for the remainder of this application, cell injury will include all cellular effects resulting from the delivery of energy from the electrodes 115,216 up to, and including, cell necrosis. Cell injury can be accomplished as a relatively simple medical procedure with local anesthesia. For example, cell injury may proceed to a depth of approximately 1-5 mms from the surface of the mucosal layer of sphincter or that of an adjoining anatomical structure.

The catheter 111 may include an insulation pad 119 that is situated between each RF electrode 115,216 and the surface of the balloon 113 for insulating and protecting the balloon 113 from the high temperatures of the RF electrode 115,216. This insulation pad 119 avoids potential damage to the catheter balloon 113 while ablative energy is effectively transmitted to the vessel surface. The insulation pad 119 also avoids potential damage to the subject's blood due to heating of the blood that has pooled behind the expanded balloon 113.

A cooling pad 119 may also be arranged between the RF electrodes 115,216 and the wire cage 114, for example so as to chill the surface of the balloon 113, thus protecting this surface from the direct effects of the RF energy, or the blood that has pooled behind the expanded balloon 113, thus protecting the subject's blood from the direct effects of the RF energy.

Also included in this first configuration design is a means to measure renal nerve afferent activity prior to and following RF nerve ablation. By measuring renal nerve activity post procedure, a degree of certainty is provided that proper nerve ablation has been accomplished. Renal nerve activity will be measured through the same mechanism as that required for energy delivery.

Nerve activity may be measured by one of two means. Proximal renal nerve stimulation will occur by means of transmitting an electrical impulse to the catheter 111 positioned within the proximal segment of the renal artery. Action potentials may be measured from the segment of the catheter 111 situated within the more distal portion of the renal artery. The quantity of downstream electrical activity as well as the time delay of electrical activity from the proximal to distal electrodes 115,216 provides a measure of residual nerve activity post nerve ablation. A second means of measuring renal nerve activity is to measure ambient electrical impulses prior to and post nerve ablation within a site more distal within the renal artery.

The RF electrodes 115,216 may operate to provide radiofrequency energy for both heating and temperature sensing. Thus, the RF elements may be used for heating during the ablation procedure and may also be used for sensing of nerve activity prior to ablation as well as after ablation has been done.

Each electrode 115,216 may be coupled to at least one sensor or control unit capable of measuring such factors as temperature, conductivity, pressure, impedance and other variables. For example, the device may have a thermistor that measures temperature in the lumen, and a thermistor may be a component of a microprocessor-controlled system that receives temperature information from the thermistor and adjusts wattage, frequency, duration of energy delivery, or total energy delivered to the electrode 115,216.

The catheter 111 may be coupled to a visualization apparatus, such as a fiber optic device, a fluoroscopic device, an anoscope, a laparoscope, an endoscope or the like. Devices coupled to the visualization apparatus may be controlled from a location outside the body, such as by an instrument in an operating room or an external device for manipulating the inserted catheter 111.

The catheter 111 may be constructed with markers that assist the operator in obtaining a desired placement, such as radio-opaque markers, etchings or microgrooves. Thus, the catheter 111 may be constructed to enhance its imageability by techniques such as ultrasounds, CAT scan or MRI. In addition, radiographic contrast material may be injected through a hollow interior of the catheter 111 through an injection port, thereby enabling localization by fluoroscopy or angiography.

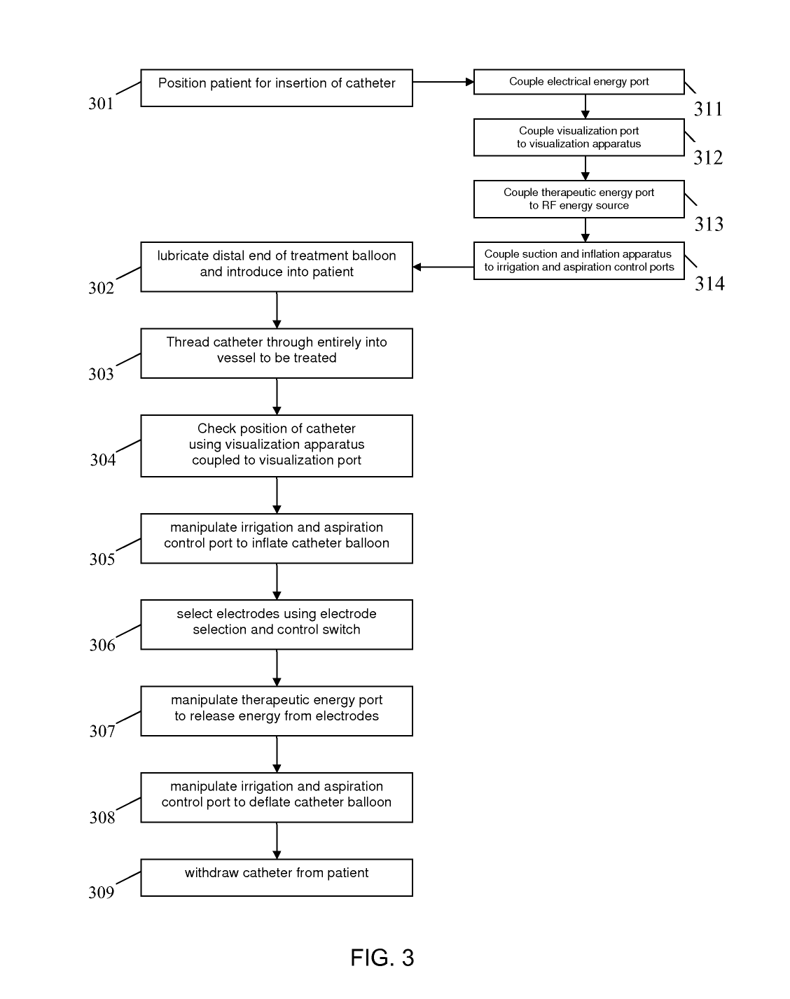

FIG. 3 is a block diagram illustrating a process for ablation of nerve function within the kidney using the devices described with FIGS. 1 and 2. The method is performed by a system including a catheter 111 and a control assembly. Although the method is described serially, the steps of the method can be performed by separate elements in conjunction or in parallel, whether asynchronously, in a pipelined manner, or otherwise. There is no particular requirement that the method be performed in the same order in which this description lists the steps, except where so indicated.

First (step 301), the patient is positioned on a treatment table in an appropriate position for the insertion of a device, and the device is prepared.

An electrical energy port is coupled to a source of electrical energy (step 311).

A visualization port is coupled (step 312) to the appropriate visualization apparatus, such as a fluoroscope, an endoscope, a display screen or other visualization device. The choice of visualization apparatus is responsive to judgments by medical personnel.

A therapeutic energy port is coupled (step 313) to the source of RF energy.

Suction and inflation apparatus are coupled (step 314) to the irrigation and aspiration control ports 117 so that the catheter balloon 113 may be later be inflated.

The most distal end of the treatment balloon 113 is lubricated and introduced into the patient (step 302). The balloon 113 may be completely deflated during insertion. The catheter 111 may be inserted into the body lumen through its outer surface, and insertion may be percutaneous or through a surgically created arteriotomy or during an open surgical procedure.

The catheter 111 is threaded through the vessel until the balloon 13 is situated entirely within the vessel to be treated (step 303). An introducer sheath or guide tube may also be used to facilitate insertion.

The position of the catheter 111 is checked using visualization apparatus coupled to the visualization port (step 304). This apparatus can be continually monitored by medical professionals throughout the procedure.

The irrigation and aspiration control port 117 is manipulated so as to inflate the balloon 113 (step 305), causing the wire frame 114 to revert to its expanded configuration, in which the wire frame 114 expands to fit within the vessel interior

Electrodes 115,216 are selected using the electrode selection and control switch (step 306). All electrodes 115,216 may be deployed at once, or electrodes 115,216 may be individually selected. This step may be repeated at any time prior to to a release of energy from the electrodes.

The therapeutic energy port 118 is manipulated so as to cause a release of energy from the electrodes 115,216 (step 307). The duration and frequency of energy are responsive to judgments by medical personnel. This release of energy creates a pattern of lesions in the lumen.

Steps 306 and 307 are repeated as many times as necessary.

The irrigation and aspiration control port 117 is manipulated so as to cause the balloon 113 to deflate and the wire frame 114 to revert to its collapsed state (step 308).

The catheter 111 may then be withdrawn from the patient (step 309).

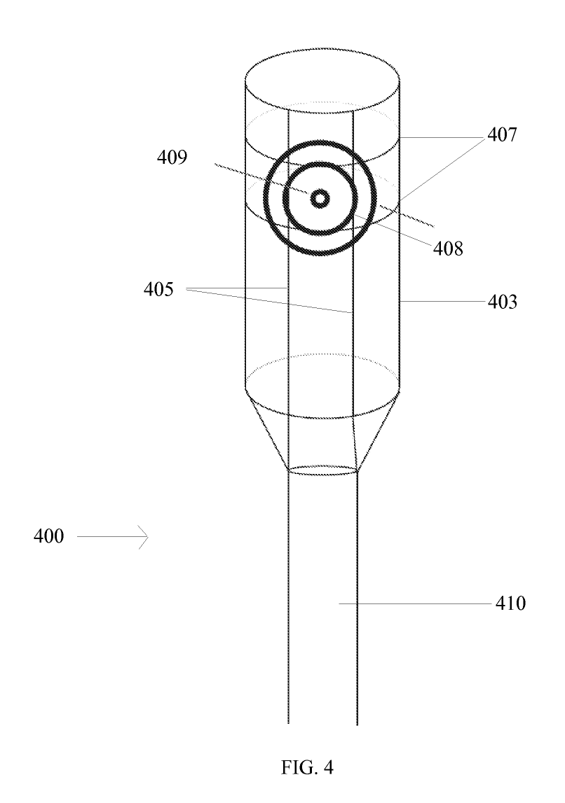

FIG. 4 illustrates a device 400 based on the second configuration for delivering radiofrequency energy to a body lumen. Radiofrequency energy may be delivered, for example, to the walls of the renal artery or aorta using a nonconductive catheter.

The device 400 includes a substantially tubular catheter (not shown), namely a long, thin, tube-like device, having proximal and distal openings, preferably constructed from a nonconductive material. The catheter can be any type of catheter, as are well known to those in the art, having a proximal end for manipulation by an operator and a distal end for operation within a patient. The distal end and proximal end preferably form one continuous piece. As will be discussed in greater detail below, the catheter is used as a delivery system for delivering a device containing radiofrequency electrodes to the desired site for nerve ablation.

As is known in the art, a guide wire 112, such as one having 0.035″ thickness, may first be inserted into the patient's vascular system via a natural orifice, a stoma or a surgically created opening that is made for the purpose of inserting the catheter, e.g. through the groin, and advanced to the desired location.

Next, a catheter is inserted into the patient and threaded over the guide wire to the desired location. The device 400 may be advanced to the desired location within the patient's vascular system with, e.g., a rapid exchange (RX) or over-the-wire wire (OTW) delivery system. Radiographic contrast media may be injected at the beginning of the procedure, e.g., through the imaging catheter port, in order to assist in manipulation of the instruments.

The device 400 comprises a wire frame or stent 114 bearing one or more electrodes 408 that are capable of conducting RF energy and that come in contact with the body tissue. The one or more electrodes 408 may have a generally circular configuration at one side of the wire frame 403. If more than one electrode 408 is used, then the circular electrodes 408 may be positioned concentrically. The wire frame 403 may be expanded so as to contact against the inner surface of the aorta at the juncture of the renal artery, such that the circular electrodes 408 are situated about the renal artery ostium.

The wire frame or stent 403 may have a generally cylindrically shaped, so that, when positioned within the aorta, its outside surfaces rest against the inner surface of the aorta. As shown in FIG. 4, the structure of the wire frame or stent 403 has two or more elongated supports 405 that are connected to two or more circular rings 407. For example, the structure of the wire frame or stent 403 may have two to four elongated supports 405, although more or fewer elongated supports 405 may be used, as necessary. Similarly, the structure of the wire frame or stent 403 may have two to four circular rings 407 positioned substantially transverse to the elongated supports 405, although more or fewer circular rings 407 may be used, as necessary. The elongated supports 405 are connected to the circular rings 407 by any method, e.g., welding.

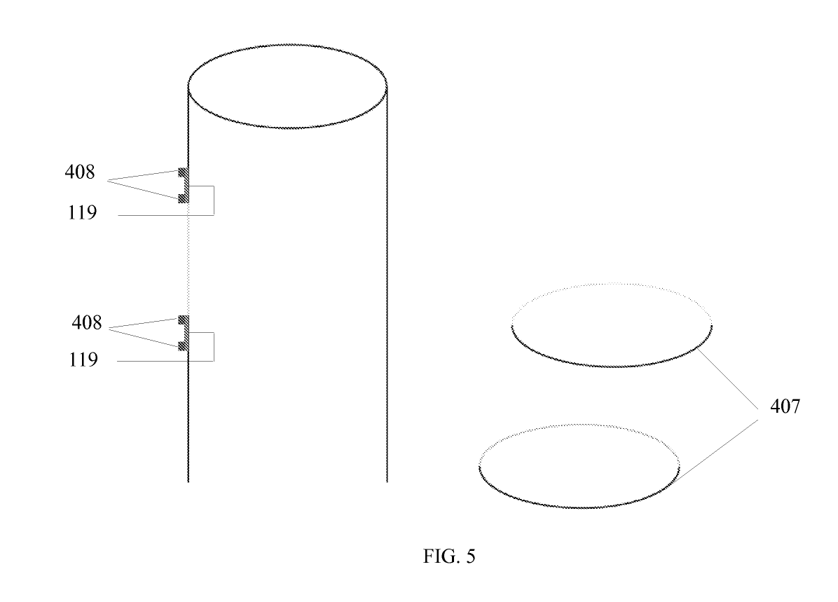

FIG. 5 shows these circular rings 407 in an exploded configuration.

The wire frame 403 may be formed from a material that is flexible and has a shape memory, e.g., nitinol. The natural shape of the wire frame 403 is in an expanded, generally cylindrical configuration, as shown in FIG. 4. In particular, the elongated supports 405 have a natural straight configuration, and the transverse rings 7 have a natural circular configuration. However, the elongated supports 405 and circular rings 407 of the wire frame 403 may be formed from a material that is sufficiently flexible and elastic so as to allow them to be flexed and deformed into other shapes, such as a collapsed configuration, upon application of an external force. The material of the wire frame 403 may have a sufficient shape memory such that the elongated supports 405 and circular rings 407 of the wire frame 403 will return to their natural configurations when the external force is released.

The wire frame or stent 403 may be selectively movable between a non-deployed position and a deployed position. In the non-deployed position, the wire frame 403 is stored unexpanded, i.e., in a collapsed configuration. The collapsed wire frame 403 in its non-deployed position may be positioned or encapsulated within a sheath 410 at the end of the catheter.

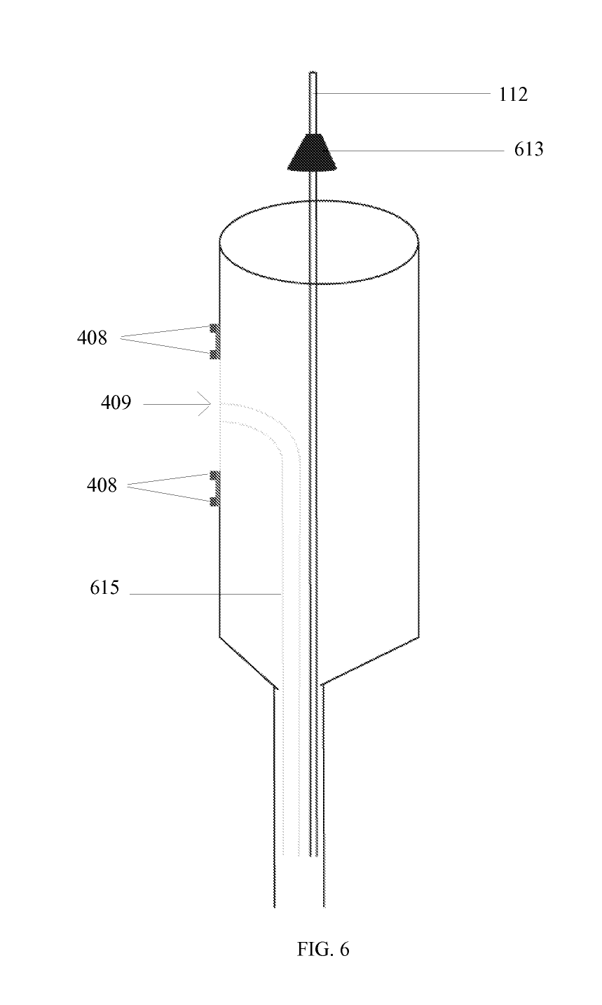

A guide wire 112 may first be inserted into the patient's vascular system via a natural orifice, e.g. through the groin, and advanced to the desired location. A cap 613 at the distal end of the guide wire 112 (see FIG. 6) facilitates entrance through the skin, and the cap and guide wire may be later separated from the sheath 410 for later deployment of the ablative elements. The catheter comprising the sheath 410 is advanced longitudinally through the blood vessel, e.g., over the guide wire 112, to the relevant location within the body lumen, such as within the aorta, and into the desired position within the inner circumference of the vessel, such as at the renal artery ostium of the aorta.

The sheath 410 is then withdrawn, thereby removing the constraint that kept the wire frame 403 in its collapsed configuration. Withdrawing the sheath 410 exposes the wire frame 403 or stent member 403, allowing it to spontaneously expand into its natural cylindrical configuration, i.e., the deployed position, wherein it conforms to the walls of the lumen.

The wire frame or stent 403 is also movable between the deployed, expanded position and a non-deployed, collapsed position. It is desirable for the wire frame 403 to be collapsible back to its non-deployed position for retraction back into the catheter sheath 410 after ablation is complete and when it is desired to withdraw the catheter from the patient.

The wire frame 403 comprises at least one electrode 408 that is capable of conducting RF energy and that comes in contact with the body tissue. There may be only one circularly shaped electrode 408, or there may be two or more circularly shaped electrodes 408. By including multiple RF electrodes in a single catheter system, more complete nerve ablation is ensured.

As shown in FIG. 4, RF electrodes 408 are attached to the wire frame 403 as a means to deliver RF energy to the body lumen, as well as temperature and nerve activity sensing. The electrodes 408 may be positioned on the outside of one side of the wire frame 403, or may be attached to the two elongated supports 405 on one side of the wire frame 403. The purpose of positioning the electrodes 408 on one side of the wire frame 403 is so that, when the wire frame 403 is expanded within the aorta and the against the insides of the aorta, the electrodes 408 would be situated on one specific side of the aorta, e.g., the side that branches off to the renal artery for more effective ablation of, e.g., the renal nerve, called the renal artery ostium.

If the RF electrodes 408 are attached to the elongated supports 405, the supports 405 may be adapted to conduct RF energy from the RF control unit to the RF electrodes 408. As such, these two elongated supports 405 serve to house connections from the RF control unit and the attached RF electrodes for temperature control and ablative energy.

When the wire frame 403 is changed into its deployed position by withdrawal of the sheath, the electrodes 408 that are positioned on the wire frame directly contact the lumen wall. If the wire frame 403 has been properly positioned before the withdrawal of the sheath 410, then the electrodes 408 contact the lumen wall at the desired location, e.g., the renal artery ostium. Heat is then generated to the electrodes 408 by supplying a suitable RF energy source to the apparatus, and the ablation is performed for the ablation of nerve activity, such as nerve activity that leads specifically to the kidney.

The device 400 may include a positioning element or mechanism for positioning and securing the device 400 at the desired location within the vessel, e.g., the aorta. Such a mechanism may ensure that the electrodes operate at a precise location, namely around the renal artery ostium. Otherwise, if the device is not properly positioned, the electrodes 408 can ablate tissue that is not intended to be harmed, causing irreversible damage. If the RF electrodes 408 are circularly shaped, the positioning mechanism may center the electrodes circumferentially around the renal artery ostium, namely the opening to the renal artery.

As shown in FIG. 6, the positioning element or mechanism may include an imaging catheter 615 that allows the user to view exactly where the renal artery ostium is and to properly position the device 400, and specifically the RF electrodes 408, through use of visual means. The imaging catheter 615 comprises a proximal end that is external to the patient and manipulated by the user along with the operating end of the device 400, and also comprises a distal end that is situated within the wire frame 403 of the device 400. The distal end of the imaging catheter 615 may extend from the proximal direction into the wire frame 403 and pass out of the wire frame 403 in a direction transverse to the longitudinal direction of the wire frame 403. For example, the distal end of the imaging catheter 615 may pass out of the wire frame 403 through the center hole 409 of the circularly-configured RF electrodes 408, as shown in FIG. 6.

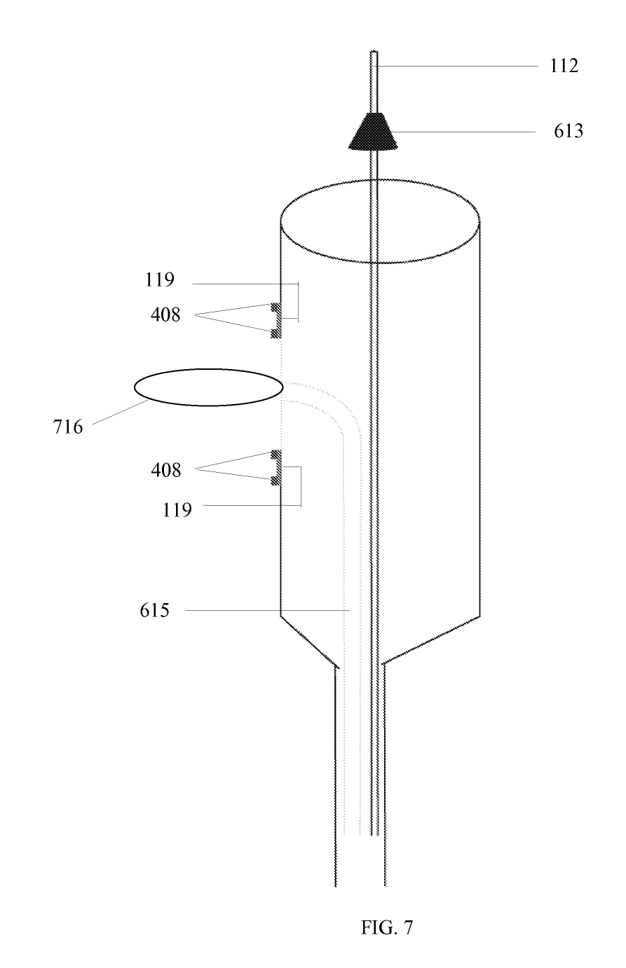

As shown in FIG. 7, the positioning element or mechanism may include a catheter that comprises an inflatable balloon 716 at its distal end that is projected into the entrance to the renal artery. This inflatable positioning balloon 716 passes through the imaging catheter 615 and the wire frame 403 from the distal direction and passes through the hole 409 of the circularly-configured RF electrodes 408, in the manner of the imaging catheter. The balloon catheter may comprise a proximal end that is external to the patient and manipulated by the user along with the operating end of the device 400, and also comprises a distal end that is situated within the wire frame 403 of the device 400. The distal end of the balloon catheter 716 extends from the proximal direction into the wire frame 403 and passes out of the wire frame 403 in a direction transverse to the longitudinal direction of the wire frame 403. For example, the distal end of the balloon catheter 716 shown in FIG. 7 passes out of the wire frame 403 through the center hole 409 of the circularly-configured RF electrodes 408.

The inflatable positioning balloon 716 is situated at the distal end of the balloon catheter. The balloon catheter 716 may be inserted at least partially into the entrance of the renal artery, and the catheter sheath 410 is then withdrawn, exposing the balloon 716 at the end thereof. The balloon is then inflated against the inner walls of the renal artery, to allow the device 400 to hold its position within the aorta relative to the renal artery. The diameter of the balloon 716, when expanded, is dependent upon the internal diameter of the branch artery at which positioning is desired. Generally, a balloon 716 with an expanded diameter of approximately 4 to 5 mm is sufficient. When the device is so positioned by virtue of the inflatable balloon 716, the wire frame can be expanded to the inner surface of the aorta, such as by retraction of the device sheath, allowing the RF electrodes 408 to be positioned against the renal artery ostium so that they may perform their ablative function.

The imaging catheter 615 and the balloon catheter 716 may both include an outer sheath 410 that is inserted into the wire frame 403 using a guide wire 112, through which sheath 410 the imaging device and the balloon device may be inserted. For example, an imaging catheter 615 may be inserted and used and then removed, leaving the sheath therefrom remaining within the patient and extending through the wire frame 403 and into the renal artery ostium. The balloon 716 may be advanced through the sheath (e.g., over a guide wire 112) and into the renal artery ostium for anchoring of the device therein. Radiographic contrast media injected at the beginning of the procedure may assist in manipulation of the instruments.

The positioning element or mechanism may operate to position the circularly-configured RF electrodes 408 at the renal artery ostium, and specifically around the opening to the branch renal artery off the ostium. This is accomplished by insertion of the distal end of the imaging catheter 615 or balloon catheter 716 that has exited the wire frame 403 of the device through the center hole 409 of the circularly-configured RF electrodes 408 at least partially into the entrance of the renal artery so as to serve, either by itself or by inflation of the balloon 716 that is exposed from within, as an anchor for the device 400 within the aorta. When the distal end of the imaging catheter 615 or the balloon 716 that is exposed from the distal end of the balloon catheter 716 is so positioned, the device 400 is able to hold its position within the aorta relative to the renal artery, and the wire frame 403 can be expanded to abut against the inner surface of the aorta. When the wire frame 403 is expanded against the inner surface of the aorta, the RF electrodes 408 can be centered circumferentially around the opening to the renal artery, i.e., the renal artery ostium, so that the RF electrodes 408 can perform their ablative function.

It should be noted that the distal end of the positioning mechanism, whether the imaging catheter 615 or the balloon catheter 716, is inserted at least partially into the entrance of the renal artery so as to serve as an anchor even before the wire frame 403 has been expanded. However, if the wire frame 403 is comprised of shape memory material such that the wire frame 403 expands spontaneously when released from the constraints that keep it in the collapsed position, the wire frame 403 may not expand until and unless the sheath 410 covering the entire device is withdrawn. Therefore, a way may be included for the positioning mechanism to protrude out of the wire frame 403 and device 400 and extend into the entrance of the renal artery so as to position the device 400 at the renal artery ostium, even while the wire frame 403 is still in its collapsed, non-deployed configuration within the sheath 410 and even while the sheath 410 has not yet been withdrawn from over the wire frame 403.

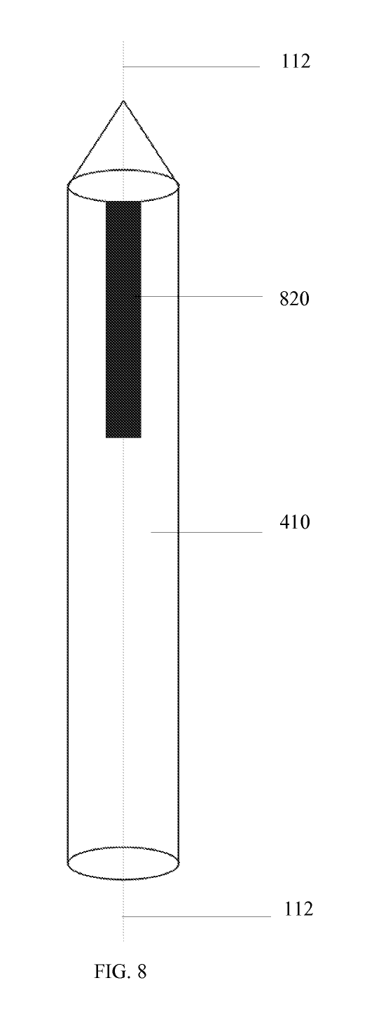

As shown in FIG. 8, the sheath 410 that envelopes the device has a longitudinal cut out 820 from its distal-most edge. This cut out 820 should be wide enough to allow the positioning device to pass through to allow the imaging catheter 615 or the balloon catheter 716 to be positioned within the entrance of the renal artery even while the sheath 410 is still in position around the wire frame 403 and keeping the wire frame in a collapsed and non-deployed position.

While the wire frame 403 is within the sheath 410, the imaging catheter 615 or the balloon catheter 716 may be manipulated to that it is positioned within the wire frame 403 but just behind the circularly-configured RF electrodes 408, as shown in cross-sectional view in FIG. 6. When it is desired for the imaging catheter 615 or the balloon catheter 716 to serve as a positioning mechanism to position the device within the aorta, the sheath is rotated about its longitudinal axis so that the cut out 820 is oriented over the center hole 409 of the circularly-configured RF electrodes 408. This exposes the center hole 409 of the circularly-configured RF electrodes 408, allowing the imaging catheter 615 or balloon catheter 716 to be pushed through the center hole 409 of the circularly-configured RF electrodes 408 and into the entrance of the renal artery.

In the case where the positioning mechanism comprises an imaging catheter 615, the device 400 is considered to be properly positioned within the aorta once the imaging catheter 715 is positioned at least partially within the entrance of the renal artery. In the case where the positioning mechanism comprises a balloon catheter 716, even if the balloon catheter 716 is positioned at least partially within the entrance of the renal artery, the device 400 is not considered to be properly positioned within the aorta until the sheath of the balloon catheter 716 is withdrawn and the balloon 716 is expanded. Once the balloon 716 is expanded within the entrance of the renal artery, the balloon catheter 716, as well as the device from which the balloon catheter 716 protrudes, is held securely therein.

Once the device 400 has been properly positioned, e.g., by insertion of the distal end of the imaging catheter 615 at least partially into the entrance of the renal artery, the sheath 410 is withdrawn or retracted, and the wire frame 403 and its attached RF electrode(s) 408 are exposed, allowing the wire frame 403 to expand. Then, if the device has been properly positioned, expansion of the wire frame 403 will result in its outer surface resting against the inside surface edges of the aorta. And, because the imaging/positioning catheter 615 has passed through the center hole 409 of the circularly-configured RF electrodes 408 and into the entrance of the renal artery, expansion of the wire cage 403 will cause the RF electrodes 408 to be positioned directly against the renal artery ostium.

At the proximal end thereof, the catheter includes at least one port. This port is for connection to a source of radiofrequency (RF) power and can be coupled to a source of Radiofrequency (RF) energy, such as RF in about the 300 kilohertz to 500 kilohertz range. The electrodes 408 are electrically coupled to the RF energy source through this port. The catheter may also be connected to a control unit for sensing and measurement of other factors, such as temperature, conductivity, pressure, impedance and other variables, such as nerve energy.

The catheter may also be connected to a second port for connection to an air source. This port would be used when it is needed for inflation and deflation of a balloon, such as in an embodiment when a balloon 716 is used in a positioning mechanism. This port can be pneumatically coupled to a pump or other apparatus to inflate or deflate the balloon. This same port may be used to circulate coolant to the inside of the balloon for the purpose of cooling the balloon during RF energy activation.

The RF electrodes 408 may operate to provide radiofrequency energy for heating of the desired location during the nerve ablation procedure. Electrodes 408 may be constructed of any suitable conductive material, as is known in the art. Examples include stainless steel and platinum alloys.

RF electrode 408 may operate in either bipolar or monopolar mode, with a ground pad electrode. In a monopolar mode of delivering RF energy, a single electrode is used in combination with an indifferent electrode patch that is applied to the body to form the other electrical contact and complete an electrical circuit. Bipolar operation is possible when two or more electrodes are used, such a two concentric electrodes. Electrodes 408 can be attached to an electrode delivery member, such as the wire frame 403, by the use of soldering or welding methods which are well known to those skilled in the art.

If the RF electrodes 408 are circular, the diameter of the circular RF electrodes 408 may be determined by the width of the aortic artery branch for which denervation is desired. If the diameter of the RF electrode 408 is smaller than the diameter of the aortic artery branch for which denervation is desired, the RF electrode 408 would not actually be in contact with tissue, and no ablation would occur. For example, when denervation is desired for the renal artery, which is approximately 6-7 mm in diameter at the ostium of the aorta, the diameter of the circular RF electrodes 408 must be at least that distance, i.e., 7 mm, in order to properly provide ablation at the renal artery ostium.

Where the device comprises two circularly-configured RF electrodes 408 that are arranged concentrically, the spacing between the two RF electrodes 408 determines the depth in the tissue to which ablation is accomplished. The farther apart the electrodes 408 are, the deeper the tissue denervation that is accomplished. For denervation of the renal artery, a spread of approximately 2-6 mm between the electrodes 408 provides sufficient depth of penetration into the tissue to accomplish the desired level of ablation such that denervation occurs. For example, in one embodiment, if the inner RF electrode 408 has a diameter of approximately 10 mm, then the outer RF electrode 408 would have a diameter of approximately 12-17 mm.

If an imaging catheter protrudes from the wire frame 403 from within the circularly-configured RF electrodes, the diameter of the RF electrodes 408 may be calculated with reference to the imaging catheter 615. For example, for an imaging catheter 615 whose distal end has a diameter of approximately 2 mm, the RF electrodes 408 that surround the imaging catheter 615 may be centered at 5 mm and 10 mm, respectively, from the center location of the imaging catheter 615.

Each electrode 408 can be disposed to treat tissue by delivering Radiofrequency (RF) energy. The radiofrequency energy delivered to the electrode has a frequency of about 5 kilohertz (kHz) to about 1 GHz. In specific embodiments, the RF energy may have a frequency of about 10 kHz to about 1000 MHz; specifically about 10 kHz to about 10 MHz; more specifically about 50 kHz to about 1 MHz; even more specifically about 300 kHz to about 500 kHz.

The electrodes 408 may be operated separately or in combination with each other as sequences of electrodes disposed in arrays. Treatment can be directed at a single area or several different areas of a vessel by operation of selective electrodes.

An electrode selection and control switch may include an element that is disposed to select and activate individual electrodes.

An RF power source may have multiple channels, delivering separately modulated power to each electrode. This reduces preferential heating that occurs when more energy is delivered to a zone of greater conductivity and less heating occurs around electrodes that are placed into less conductive tissue. If the level of tissue hydration or the blood infusion rate in the tissue is uniform, a single channel RF power source may be used to provide power for generation of lesions relatively uniform in size.

RF energy delivered through the electrodes 408 to the tissue causes heating of the tissue due to absorption of the RF energy by the tissue and ohmic heating due to electrical resistance of the tissue. This heating can cause injury to the affected cells and can be substantial enough to cause cell death, a phenomenon also known as cell necrosis. Cell injury may include all cellular effects resulting from the delivery of energy from the electrodes up to, and including, cell necrosis. Cell injury can be accomplished as a relatively simple medical procedure with local anesthesia. For example, cell injury may proceed to a depth of approximately 1-5 mms from the surface of the mucosal layer of sphincter or that of an adjoining anatomical structure.

As shown in FIG. 5, the catheter may include an insulation pad 119 that is situated between each RF electrode 408 and the wire frame 403, for example so as to protect the wire frame 403 from the direct effects of the RF energy. This insulation pad 119 may also avoid potential damage to the body to the subject's blood while ablative energy is effectively transmitted to the vessel surface and the blood that has passes through the wire frame.

Also included in this second configuration design is a means to measure renal nerve afferent activity prior to and following RF nerve ablation. By measuring renal nerve activity post procedure, a degree of certainty is provided that proper nerve ablation has been accomplished. Renal nerve activity may be measured through the same mechanism as that required for energy delivery and electrodes on the renal artery placed positioning balloon.

Nerve activity may be measured by one of two means. Proximal renal nerve stimulation will occur by means of transmitting an electrical impulse to the catheter positioned within the proximal segment of the renal artery. Action potentials may be measured from the segment of the catheter situated within the more distal portion of the renal artery. The quantity of downstream electrical activity as well as the time delay of electrical activity from the proximal to distal electrodes provides a measure of residual nerve activity post nerve ablation. A second means of measuring renal nerve activity is to measure ambient electrical impulses prior to and post nerve ablation within a site more distal within the renal artery.

The RF electrodes operate 408 may operate to provide radiofrequency energy for both heating and temperature sensing. Thus, the RF elements may be used for heating during the ablation procedure and may also be used for sensing of nerve activity prior to ablation as well as after ablation has been done.

Each electrode 408 may be coupled to at least one sensor or control unit capable of measuring such factors as temperature, conductivity, pressure, impedance and other variables. For example, the device may have a thermistor that measures temperature in the lumen, and a thermistor may be a component of a microprocessor-controlled system that receives temperature information from the thermistor and adjusts wattage, frequency, duration of energy delivery, or total energy delivered to the electrode.

The catheter may be coupled to a visualization apparatus, such as a fiber optic device, a fluoroscopic device, an anoscope, a laparoscope, an endoscope or the like. Devices coupled to the visualization apparatus may be controlled from a location outside the body, such as by an instrument in an operating room or an external device for manipulating the inserted catheter.

The catheter may be constructed with markers that assist the operator in obtaining a desired placement, such as radio-opaque markers, etchings or microgrooves. Thus, the catheter may be constructed to enhance its imageability by techniques such as ultrasounds, CAT scan or MRI. In addition, radiographic contrast material may be injected through a hollow interior of the catheter through an injection port, thereby enabling localization by fluoroscopy or angiography.

A method for ablation of renal artery nerve function within the aorta using the device 400 may be performed by a system including a catheter and a control assembly. Although the method will be described serially, the steps of the method can be performed by separate elements in conjunction or in parallel, whether asynchronously, in a pipelined manner, or otherwise. There is no particular requirement that the method be performed in the same order in which this description lists the steps, except where so indicated.

Referring back to FIG. 3, an electrical energy port is coupled to a source of electrical energy (step 311). The patient is positioned on a treatment table in an appropriate position for the insertion of a catheter (step 301).

The visualization port is coupled to the appropriate visualization apparatus (step 312), such as a fluoroscope, an endoscope, a display screen or other visualization device. The choice of visualization apparatus is responsive to judgments by medical personnel.

The therapeutic energy port is coupled to the source of RF energy (step 313).

Suction and inflation apparatus are coupled to the irrigation and aspiration control ports so that a catheter balloon may be later be inflated (step 314), if the balloon 716 is to be used.

The most distal end of the treatment balloon is lubricated and introduced into the patient (step 302). Preferably, the balloon is completely deflated during insertion. The catheter may be inserted into the body lumen through its outer surface, and insertion may be percutaneous or through a surgically created arteriotomy or during an open surgical procedure.

The catheter, including the wire frame and positioning device, i.e., imaging or balloon catheter, is threaded through the vessel until the wire frame is situated entirely within the vessel to be treated (step 303). An introducer sheath or guide tube may also be used to facilitate insertion.

The position of the catheter is checked using visualization apparatus coupled to the visualization port (step 304). This apparatus can be continually monitored by medical professionals throughout the procedure.

A positioning mechanism is positioned such that it protrudes through the circular electrodes into the ostium of the renal or another artery (not shown).

The irrigation and aspiration control port is manipulated so as to inflate the balloon of the positioning mechanism, causing the catheter top be rendered stable in its position within the lumen (step 305).

The device sheath is retracted, causing the wire frame to revert to its expanded configuration, in which the wire frame expands to fit within the vessel interior (not shown).

The electrodes 408 are selected using the electrode selection and control switch (step 306). Preferably, all electrodes are deployed at once, although the electrodes may be individually selected. This step may be repeated at any time prior to a release of energy from the electrodes.

The therapeutic energy port is manipulated so as to cause a release of energy from the electrodes 408 (step 307). The duration and frequency of energy are responsive to judgments by medical personnel. This release of energy creates a circular pattern of lesions at the renal artery ostium.

The device sheath is advanced over the wire frame so as to cause the wire frame to revert to its collapsed state (not shown).

The irrigation and aspiration control port is manipulated so as to cause the positioning device balloon to deflate (step 308).

The positioning device, either the balloon catheter or the imaging catheter, is withdrawn from the renal artery ostium, into the device 400 (not shown).

Once ablation is completed and the wire frame, the balloon and the imaging/positioning catheters are withdrawn into the sheath, the device is available for positioning at another location within the patient, e.g., the contralateral (or accessory) renal artery, and the steps above may be repeated for each ablation site.

The catheter may then be withdrawn from the patient (step 309).

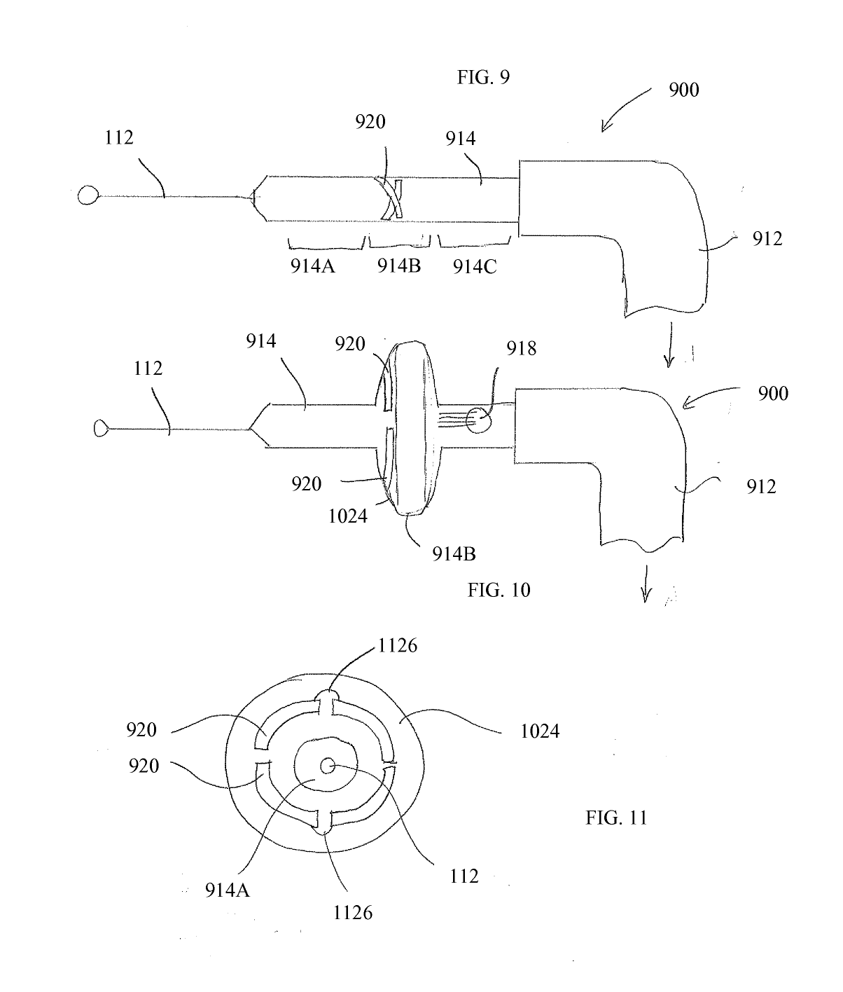

FIG. 9 is a side view drawing of a device 900 based on the third configuration for delivering radiofrequency energy to the walls of a body lumen. Radiofrequency energy may be delivered, for example, to the walls of the renal artery or aorta using a nonconductive catheter.

The device 900 includes a substantially tubular catheter 912, called a guiding catheter, namely a long, thin, tube-like device, having proximal and distal openings, preferably constructed from a nonconductive material. The guiding catheter 912 can be any type of catheter, as are well known to those in the art, having a proximal end for manipulation by an operator and a distal end for operation within a patient. The distal end and proximal end preferably form one continuous piece. As will be discussed in greater detail below, guiding catheter 912 is used as a delivery system for delivering a balloon catheter bearing radiofrequency electrodes to the desired site for nerve ablation.

A device 900 also comprises a balloon catheter 914, e.g., cylindrically shaped, that is formed of a material, such as a polymer, as is well known in the art that allows it to be expanded at some portions along its length through inflation. The balloon catheter 914, when in a non-deployed configuration, has an outer diameter that is smaller than the inner diameter of guiding catheter 912 so as to allow balloon catheter 914 to pass easily through guiding catheter 912 into the patient. The balloon catheter 914 may move within and relative to guiding catheter 912 with low friction, such that guiding catheter 912 can be retracted from balloon catheter 914 at the appropriate time.

The balloon catheter 914, as is known in the art, has a small diameter annulus therethrough to allow it to be threaded over a guide wire 112 and advanced into the patient, e.g., through guiding catheter 912. As is known in the art, the guide wire 112, such as one having 0.035″ thickness, may first be inserted into the patient's vascular system, e.g. through the groin, and advanced to the desired location. Next, the tube-like guiding catheter 912 is inserted into the patient and threaded over the guide wire 112 to the desired location. Preferably, the device 900 is advanced to the desired location within the patient's vascular system with, e.g., a rapid exchange (RX) or over-the-wire wire (OTW) delivery system with a 0.035″ or smaller guide wire 112 that is employed for the device. Radiographic contrast media may be injected at the beginning of the procedure to assist in manipulation and positioning of the instruments.

Balloon catheter 914, in an unexpanded condition, is advanced longitudinally through the blood vessel, e.g., over guide wire 112, through guiding catheter 912 to the relevant location within the body lumen, such as within the aorta, and into the desired position within the inner circumference of the vessel, such as at the renal artery ostium of the aorta. Balloon catheter 914 in its unexpanded, non-deployed position may be positioned or encapsulated within a guiding catheter 912, which functions as a retractable sheath at the end of device 900.

The balloon catheter 914 may be a noncompliant catheter that generally does not expand but has one or more different compliant sections along its length, with each section having a different level of compliancy, to allow certain portions thereof to be expanded through inflation more than other portions thereof. For example, as shown in FIG. 9, the balloon catheter 914 has the sections 914A, 914B and 914C along its distal end, with each of the sections 914A, 914B and 914C having a different level of compliancy. The section 914B of balloon catheter 914 may be formed of a very compliant material that may be expanded, while sections 914A and 914C of balloon catheter 914 may be formed of a very non-compliant material that it essentially non-expandable. The materials of balloon catheter 914 sections 914A, 914B and 914C may be bonded together to form one unitary balloon catheter device 914.

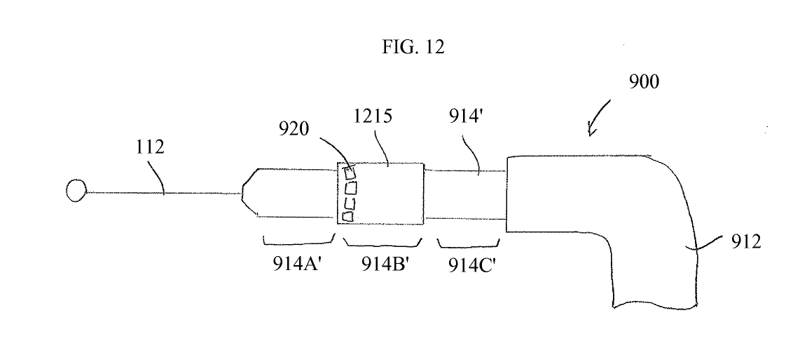

As an alternative approach, the balloon catheter may also be a noncompliant catheter that generally does not expand but has one or more separate compliant portions overlying (as a sleeve or overlay) the noncompliant catheter, with the overlying compliant portions separately or individually expandable through inflation. Referring to FIG. 12, the entire balloon catheter 914′ is formed of a very non-compliant material that is essentially non-expandable (although the base catheter can no longer truly be referred to as a “balloon” catheter since it does not expand as a balloon does). However, balloon catheter 914′ has a portion, i.e., section 914B′ between sections 914A′ and 914C′, near its distal end, that is overlaid with an annular, sleeve-like balloon overlay 1215, that is formed of a very compliant material and may be expanded.

The design principles of the balloon catheter of FIGS. 9 and 12 may also be combined. For example, such a balloon catheter is a noncompliant catheter that generally does not expand but has one or more different compliant sections along its length, with each section having a different levels of compliancy (e.g., like 914A to 914C in FIG. 9), to allow certain portions thereof to be expanded through inflation more than other portions thereof, and also has one or more separate compliant portions overlying the catheter, which overlying compliant portions may be separately or individually expandable through inflation (such as balloon portion 1215 in FIG. 12).

The balloon catheter 914 and 914′ are selectively movable between a non-deployed, unexpanded condition and a deployed, expanded condition, and back to the non-deployed, unexpanded condition. In the non-deployed condition, as shown in FIGS. 9 and 12, the balloon catheter 914/914′ of device 900 is unexpanded, i.e., in a collapsed configuration, and may be advanced longitudinally through the blood vessel, e.g., over guide wire 112 and through guiding catheter 912, to the relevant location within the body lumen, such as within the aorta, and into the desired position within the inner circumference of the vessel, such as at the renal artery ostium of the aorta. Once at the desired position, guiding catheter 912 may be retracted, revealing balloon catheter 914/914′.

The balloon catheter 914/914′, once guiding catheter 912 has been retracted, may be expanded into its deployed position for operation within the patient. In the deployed condition, as shown in FIGS. 10 and 11, the expandable portions of balloon catheter 914/914′ are expanded. The balloon catheter 914/914′ may have a port 1018, as is known in the art, through which air (or another gas) may be introduced to enable inflation of its inflatable portions.

The largest diameter of balloon catheter 914/914′ in its deployed condition is larger than the inner diameter of guiding catheter 912, such that balloon catheter 914/914′ cannot be expanded into its deployed condition while still encased within guiding catheter 912, and such that balloon catheter 914/914′ in its deployed condition cannot be retracted back into guiding catheter 912. It is desirable for balloon catheter 914/914′ to be deflated back to its non-deployed position for retraction back into the guiding catheter 912 after ablation is complete and when it is desired to withdraw the device from the patient.

When balloon catheter 914 is in its deployed position, as shown in FIG. 10, the compliant segment of balloon catheter 14 (section 14B in FIG. 9), called the balloon segment, is expanded to have a much larger diameter than the non-compliant segments 914A and 914C, such that the balloon segment 914B has a disk-like configuration with a circular, somewhat planar surface 1024 that is oriented orthogonally to the direction of guide wire 112 and facing in a distal direction. It is this distally-facing surface 1024 of the expanded balloon segment 914B that provides the ablating surface when contacting the renal artery ostium of the aorta.

Shown in its non-deployed, unexpanded condition in FIG. 12, when the balloon catheter 914′ is expanded into its deployed position, similar to as shown in FIG. 10, separately compliant annular balloon portion 1215 that overlays section 914B′ of the balloon catheter 914′ in FIG. 12, called the balloon overlay, is expanded to have a much larger diameter than the non-compliant segments 914A′ and 914C′, such that the balloon overlay 1215 has a disk-like configuration with a circular, somewhat planar surface that is oriented orthogonally to the direction of the guide wire 112 and facing in a distal direction, similar to as shown in FIG. 10. It is this distally-facing surface of the expanded balloon overlay 1215 that provides the ablating surface when contacting the renal artery ostium of the aorta.

The balloon catheter 914/914′ of the device 900 comprises one or more electrodes 920 that are capable of conducting RF energy and that come in contact with the body tissue. One or more electrodes 920 may positioned in a circular configuration on a portion of balloon catheter 914/914′ when device 900 is in its deployed position, such that electrodes 920 provide essentially 360° coverage at the renal artery ostium. If more than one electrode 920 is used, then electrodes 920 may be positioned such that, when device 900 is in a deployed position, electrodes 920 together have a circular configuration or are oriented concentrically, such that they together provide essentially 360° coverage around a target area.

When the balloon catheter 914 is in its deployed position, one or more electrodes 920 are situated on the balloon segment 914B of the device 900 when the device 900 is in its deployed position, i.e., on the distally-facing surface 1024 of the expanded balloon segment 914B (or of the expanded balloon overlay 1215 in FIG. 12), as shown in FIGS. 10 and 11. This distally-facing surface 1024 of the balloon segment 914B can be pressed up against and contacted with the inner surface of the aorta at the juncture of the renal artery, such that electrodes 920 that may be positioned, e.g., in a circular configuration, would be situated about the renal artery ostium of the aorta. When electrodes 920 are contacted against the inner surface of the lumen, e.g., the aorta, for example, at the renal artery ostium, electrodes 920 ablate the nerve activity circumferentially around the renal artery ostium.

As shown in FIG. 9, RF electrodes 920 are attached to balloon catheter 914 as a means to deliver RF energy to the body lumen, as well as temperature and nerve activity sensing. The device 900 may have several RF electrodes 920 that are attached to the surface of balloon catheter 914 separately but that, when oriented together in a deployed configuration, are positioned in a circular configuration on the distally-facing surface 1024 of the balloon segment 914B. For example, as shown in FIG. 11, the device 900 may include four arc-shaped electrodes 920. The electrodes 920 may be attached to and positioned on the outside of balloon catheter 914 at segment 914B, or as shown in FIG. 12, the electrodes 920 may be attached to and positioned on balloon overlay 1215.

When balloon catheter 914 is in its non-deployed configuration, RF electrodes 920 lie substantially flat against the surface of balloon catheter 914 and have a relatively low profile there against. Electrodes 920 may be attached to the surface of the balloon segment of the balloon catheter, e.g., by gluing, bonding, or a wire cage attachment. Thus, when balloon catheter 914 is advanced distally through guiding catheter 912 for use within the patient, or when balloon catheter 914 is advanced proximally through guiding catheter 912 for withdrawal from the patient, RF electrodes 920 do not interfere with or impede the progress of balloon catheter 914 through guiding catheter 912.

When balloon catheter 914 is in its non-deployed configuration, as shown in FIG. 9, the four arc-shaped electrodes 920 are in an overlapping relationship with respect to each other. Then, when balloon catheter 914 is expanded into its deployed configuration, the four arc-shaped electrodes 920 slide or glide past each other and become oriented into a circular configuration, as shown in FIG. 11. In this configuration, the electrodes 920 may also have an attachment means that loosely connects them to the surface of balloon catheter 914 and assists in rearranging them back into their resting configuration when balloon catheter 914 is deflated into its non-deployed configuration. The attachment also insures proper fixation of electrodes 920 to the surface of balloon catheter 914. An example of such attachment means is illustrated in FIG. 11 in the form of a shape memory wire 1126 that helps reposition electrodes 920 to the surface of the balloon segment 914B of balloon catheter 914 with respect to each other when balloon catheter 914 is deflated.

There may be one or more elongated wires (not shown) that run along the side of balloon catheter 914 to which RF electrodes 920 are attached to conduct RF energy from an external RF control unit to RF electrodes 920. All the RF electrodes 920 may be attached to the same wire such that they are made to operate together. The electrodes 920 may also have wires that loosely connect them, in order for them to be connected electrically. There may also be multiple wires, each of which is attached to as few as one electrode 920 so as to conduct RF energy from the RF control unit to the individual RF electrodes 920. The RF electrodes 920 can deliver their energy simultaneously or can deliver energy in a sequential or other desired pattern.

When balloon catheter 914 is changed into its deployed position by inflation, electrodes 920 that are positioned on the surface of balloon catheter 914 become situated on the distally-facing surface 1024 of the balloon segment 914B. The purpose of positioning electrodes 920 on one side of the distally-facing surface 1024 of the balloon segment 914B is so that electrodes 920 could be positioned or pressed up against the renal artery ostium, for more effective ablation of, e.g., the renal nerve. Guiding catheter 912 is advanced distally such that its distal edge presses against the proximally-facing surface of the expanded balloon segment 914B, thereby allowing RF electrodes 920 on the distally-facing surface 1024 of the expanded balloon segment 914B to be pushed distally and positioned against the renal artery ostium so that they may perform their ablative function. When electrode-bearing distally-facing surface 1024 of the balloon segment 914B is pressed up against the renal artery ostium of the aorta, electrodes 920 that are positioned in a circular configuration may be made to contact the renal artery ostium of the aorta. Heat is then generated to electrodes 920 by supplying a suitable RF energy source to device 900, and the ablation is performed for the ablation of nerve activity, such as nerve activity that leads specifically to the kidney.

The device 900 may have a positioning element or mechanism for positioning and securing device 900 at the desired location within the vessel, e.g., the aorta. Such a mechanism may ensure that the electrodes 20 operate at a precise location, namely around the renal artery ostium. Otherwise, if device 900 is not properly positioned, electrodes 920 can ablate tissue that is not intended to be harmed, causing irreversible damage. If the RF electrodes 920 are circularly configured, the positioning mechanism may center the electrodes 920 circumferentially around the renal artery ostium, namely the opening to the renal artery.

Such a positioning mechanism may include, for example, guide wire 112 and the distal, unexpanded section 914A of balloon catheter 914 that is inserted at least partially into the entrance to the renal artery and remains there. Once this is done, guiding catheter 912 overlying balloon catheter 914 is withdrawn proximally, and balloon segment 914B of balloon catheter 914 may then be inflated. Guiding catheter 912 may then be advanced distally such that its distal edge presses against the proximally-facing surface of expanded balloon segment 914B, thereby allowing RF electrodes 920 on the distally-facing surface 1024 of expanded balloon segment 914B to be positioned against the renal artery ostium so that they may perform their ablative function.

The positioning mechanism may include a separately compliant portion of balloon catheter 914, namely the section 914A of balloon catheter 14 that is situated distally of balloon segment 914B. The section 914A of balloon catheter 914 may be separately inflatable, and, because it projects into the entrance to the renal artery, is called the positioning segment. This positioning segment 914A of balloon catheter 914 is inserted at least partially into the entrance of the renal artery and is then inflated, not to the extent of the balloon segment 914B but only approximately to the diameter of the renal artery, so as to prevent the balloon catheter 914 from being moved distally or proximally relative to the renal artery, so as to allow the device 900 to hold its position within the renal artery relative to the aorta. When the device 900 is so positioned by virtue of the inflatable balloon in the positioning segment 914A of the balloon catheter 914, circularly configured RF electrodes 920 may be positioned against the renal artery ostium so that they may perform their ablative function. Before the positioning segment 914A of balloon catheter 914 is expanded, the distal edge of the guiding catheter 912 presses against the proximally-facing surface of the expanded balloon segment 914B, thereby allowing RF electrodes 920 on the distally-facing surface 1024 of expanded balloon segment 914B to be positioned against the renal artery ostium.

The positioning mechanism may comprise both an unexpanded section of balloon catheter 914 at its distal end and a separately inflatable portion that is situated distally of the balloon segment. The unexpanded section of the balloon catheter 914 may be inserted at least partially into the entrance to the renal artery to help guide the device to the correct location in the aorta, and the separately inflatable portion of the balloon catheter 914 may be inflated within the renal artery so to hold the device in its position within the renal artery relative to the aorta.

The positioning mechanism may include an imaging catheter at the distal end of balloon catheter 914 that allows the user to view exactly where the renal artery ostium is and to properly position the device within the renal artery, through use of visual means. The imaging catheter may comprise a proximal end that is external to the patient and manipulated by the user along with the operating end of the device, and also comprises a distal end that is situated at the distal end of the balloon catheter 914.

The positioning element or mechanism operates to position balloon catheter 914 within the renal artery so that the circularly-configured RF electrodes 920 can be pressed against the renal artery ostium, and specifically around the opening to the branch renal artery off the ostium. This may be accomplished by insertion of the unexpanded distal end of the balloon catheter 914 or the distal end of the imaging catheter at least partially into the entrance of the renal artery so as to serve, either by itself or by inflation of a balloon that is exposed from within, as an anchor for the device 900 within the aorta so that RF electrodes 920 can perform their ablative function.

At the proximal end thereof, device 900 may include at least one port 1018 for connection to a source of radiofrequency (RF) power. Device 900 may be coupled to a source of Radiofrequency (RF) energy, such as RF in about the 300 kilohertz to 500 kilohertz range. The electrodes may be electrically coupled to the RF energy source through this port. Device 900 may be coupled to a source of air for inflation of the inflatable portions of balloon catheter 914. Device 900 may also be connected to a control unit for sensing and measurement of other factors, such as temperature, conductivity, pressure, impedance and other variables, such as nerve energy.

Device 900 may also be connected, either through port 1018 or through a second port, to an air or fluid source. This port can be pneumatically or hydraulically coupled to a pump or other apparatus for inflation and deflation of the inflatable portions of balloon catheter 914. The port may also be used for inflation and deflation of the balloon overlay of balloon catheter 914′, when it is present. The port may further be used for inflation and deflation of a balloon used in a positioning mechanism. There may be one port for all balloons or separate ports for one or more balloons. This same port may be used to circulate coolant to the inside of the balloon for the purpose of cooling the balloon during RF energy activation.

The RF electrodes 20 may operate to provide radiofrequency energy for heating of the desired location during the nerve ablation procedure. The electrodes 920 may be constructed of any suitable conductive material, as is known in the art. Examples include stainless steel and platinum alloys.

The RF electrodes 920 may operate in either bipolar or monopolar mode, with a ground pad electrode. In a monopolar mode of delivering RF energy, a single electrode is used in combination with an indifferent electrode patch that is applied to the body to form the other electrical contact and complete an electrical circuit. Bipolar operation is possible when two or more electrodes are used, such a two concentric electrodes. The electrodes 920 may be attached to an electrode delivery member, such as the wire frame, by the use of soldering or welding methods which are well known to those skilled in the art.

If one or more arc-shaped RF electrodes 920 are oriented in a circular configuration, the diameter of the circular or arc-shaped RF electrodes 920 may be determined by the width of the aortic artery branch for which denervation is desired. If the diameter of the RF electrode is smaller than the diameter of the aortic artery branch for which denervation is desired, the RF electrode would not actually be in contact with tissue, and no ablation would occur. For example, when aortic denervation is desired at the level of the renal artery ostium, which is approximately 6-7 mm in diameter at the ostium of the aorta, the diameter of the circular RF electrodes must be at least that distance, i.e., 7 mm, in order to properly provide ablation surrounding the renal artery ostium. The length of each of four arc-shaped electrodes 920 may be, for example, approximately 2-3 mm.

The diameter of RF electrodes 920 may be calculated with reference to the renal artery ostium. For example, if it is desired that the RF energy be applied at least approximately 2 mm from each edge of the renal artery ostium, the RF electrodes that surround the imaging catheter may have a 10-14 mm diameter surrounding the renal artery ostium.

Each electrode 920 can be disposed to treat tissue by delivering radiofrequency (RF) energy. The radiofrequency energy delivered to the electrode has a frequency of about 5 kilohertz (kHz) to about 1 GHz. In specific embodiments, the RF energy may have a frequency of about 10 kHz to about 1000 MHz; specifically about 10 kHz to about 10 MHz; more specifically about 50 kHz to about 1 MHz; even more specifically about 300 kHz to about 500 kHz.

The electrodes 920 may be operated separately or in combination with each other as sequences of electrodes disposed in arrays. Treatment can be directed at a single area or several different areas of a vessel by operation of selective electrodes.

An electrode selection and control switch may include an element that is disposed to select and activate individual electrodes.

An RF power source may have multiple channels, delivering separately modulated power to each electrode. This reduces preferential heating that occurs when more energy is delivered to a zone of greater conductivity and less heating occurs around electrodes that are placed into less conductive tissue. If the level of tissue hydration or the blood infusion rate in the tissue is uniform, a single channel RF power source may be used to provide power for generation of lesions relatively uniform in size.

RF energy delivered through the electrodes to the tissue causes heating of the tissue due to absorption of the RF energy by the tissue and ohmic heating due to electrical resistance of the tissue. This heating can cause injury to the affected cells and can be substantial enough to cause cell death, a phenomenon also known as cell necrosis. Cell injury may include all cellular effects resulting from the delivery of energy from the electrodes up to, and including, cell necrosis. Cell injury can be accomplished as a relatively simple medical procedure with local anesthesia. For example, cell injury may proceed to a depth of approximately 1-5 mms from the surface of the mucosal layer of sphincter or that of an adjoining anatomical structure.

The balloon catheter 914 may further comprise an insulation pad that is situated between each RF electrode 920 and the surface of balloon catheter 914, for example so as to protect balloon catheter 914 from the direct effects of the RF energy. The balloon catheter 914 also may contain a circulating coolant so as to cool the balloons and protect it from the direct effects of the RF energy.

Also included in this third configuration design is a means to measure renal nerve afferent activity prior to and following RF nerve ablation. By measuring renal nerve activity post procedure, a degree of certainty is provided that proper nerve ablation has been accomplished. Renal nerve activity may be measured through the same mechanism as that required for energy delivery and electrodes on the renal artery placed positioning balloon.

Nerve activity may be measured by one of two means. Proximal renal nerve stimulation will occur by means of transmitting an electrical impulse to the catheter positioned within the proximal segment of the renal artery. Action potentials may be measured from the segment of the catheter situated within the more distal portion of the renal artery. The quantity of downstream electrical activity as well as the time delay of electrical activity from the proximal to distal electrodes provides a measure of residual nerve activity post nerve ablation. A second means of measuring renal nerve activity is to measure ambient electrical impulses prior to and post nerve ablation within a site more distal within the renal artery.

The RF electrodes 920 may operate to provide radiofrequency energy for both heating and temperature sensing. Thus, the RF elements may be used for heating during the ablation procedure and also be used for sensing of nerve activity prior to ablation as well as after ablation has been done.

Each electrode 920 may be coupled to at least one sensor or control unit capable of measuring such factors as temperature, conductivity, pressure, impedance and other variables. For example, the device may have a thermistor that measures temperature in the lumen, and a thermistor may be a component of a microprocessor-controlled system that receives temperature information from the thermistor and adjusts wattage, frequency, duration of energy delivery, or total energy delivered to the electrode.

The device 900 may be coupled to a visualization apparatus, such as a fiber optic device, a fluoroscopic device, an anoscope, a laparoscope, an endoscope or the like. Devices coupled to the visualization apparatus may be controlled from a location outside the body, such as by an instrument in an operating room or an external device for manipulating the inserted catheter.

The device 900 may be constructed with markers that assist the operator in obtaining a desired placement, such as radio-opaque markers, etchings or microgrooves. Thus, device 900 may be constructed to enhance its imageability by techniques such as ultrasounds, CAT scan or MRI. In addition, radiographic contrast material may be injected through a hollow interior of the catheter through an injection port, thereby enabling localization by fluoroscopy or angiography.

A method for ablation of renal artery nerve function within the aorta using the device 900 may be performed by a system including a device 10 and a control assembly (not shown). Although the method is described serially, the steps of the method can be performed by separate elements in conjunction or in parallel, whether asynchronously, in a pipelined manner, or otherwise. There is no particular requirement that the method be performed in the same order in which this description lists the steps, except where so indicated.

Referring back to FIG. 3, an electrical energy port is coupled to a source of electrical energy (step 311). The patient is positioned on a treatment table in an appropriate position for the insertion of a catheter (step 301).

The visualization port is coupled to the appropriate visualization apparatus (step 312), such as a fluoroscope, an endoscope, a display screen or other visualization device. The choice of visualization apparatus is responsive to judgments by medical personnel.

The therapeutic energy port is coupled to the source of RF energy (step 313).

Suction and inflation apparatus are coupled to the irrigation and aspiration control ports so that a catheter balloon may be later be inflated (step 314).

The guide wire 112 and guiding catheter 912 or tube are lubricated and introduced into the patient (similar to step 303). Insertion may be percutaneous or through a surgically created arteriotomy or during an open surgical procedure.

The most distal end of balloon catheter 914 is lubricated and introduced into the patient (step 302). Preferably, the balloon is completely deflated during insertion. Balloon catheter 914 may be inserted into the body lumen through its outer surface and is threaded through the vessel until the balloon portion is situated adjacent to the vessel to be treated.

The position of the device 900 is checked using visualization apparatus coupled to the visualization port (step 304). This apparatus can be continually monitored by medical professionals throughout the procedure.

A positioning mechanism, if used, is positioned such that it protrudes into the ostium of the renal or another artery (not shown).

The guiding catheter 12 is retracted, allowing balloon catheter 14 to be expanded (not shown).

The irrigation and aspiration control ports are manipulated so as to inflate the balloon of the positioning mechanism, causing device 900 to be rendered stable in its position within the lumen, and so as to inflate the balloon segment 914B of balloon catheter 914 (step 305).

The guiding catheter 912 is advanced distally so that its distal-most edge presses against the proximally-facing surface of the expanded balloon segment 914B and pushing the distally-facing surface 1024 of the expanded balloon segment 914B against the renal artery ostium (not shown).

The electrodes 920 on the distally-facing surface 1024 of the expanded balloon segment 914B are selected using the electrode selection and control switch (step 306). Preferably, all the electrodes 920 are deployed at once. Also preferably, the electrodes 920 may be individually selected. This selection of electrodes may be repeated at any time prior to a release of energy from the electrodes.

The therapeutic energy port is manipulated so as to cause a release of energy from electrodes 920 (step 307). The duration and frequency of energy are responsive to judgments by medical personnel. This release of energy creates a circular pattern of lesions at the renal artery ostium.

The irrigation and aspiration control port is manipulated so as to cause the positioning device balloon and balloon segment 914B to deflate (step 308).

The guiding catheter 912 is advanced over deflated balloon catheter 914 (not shown).

The positioning device and balloon catheter 914 are withdrawn from the renal artery ostium, into guiding catheter 912.

The guiding catheter 912 may then be withdrawn from the patient (step 309).

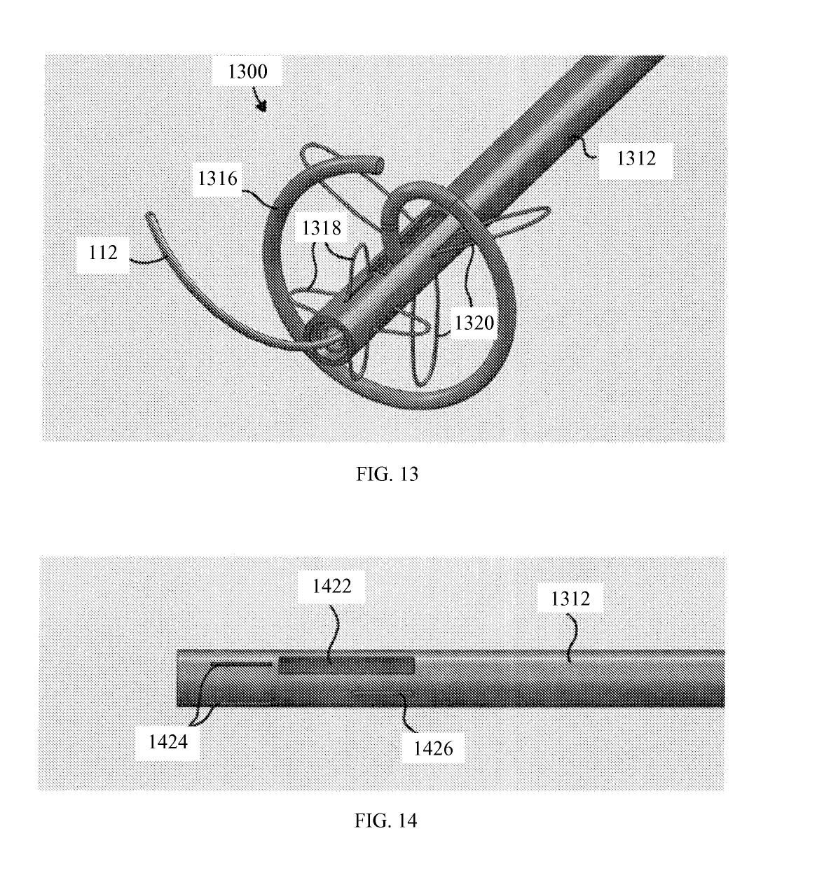

FIG. 13 illustrates an ablation device 1300 based on the fourth configuration for delivering radiofrequency energy to the walls of a body lumen. The device is used for interaortic renal artery ablation for renal artery sympathetic neural ablation. Radiofrequency energy may be delivered, for example, to the walls of the renal artery or aorta using a nonconductive catheter.

The device 1300 includes a substantially tubular catheter 1312, called a delivery catheter, namely an elongated, thin, tube-like device, having proximal and distal ends, preferably constructed from a nonconductive material. The delivery catheter 1312 can be any type of catheter, as are well known to those in the art, having a proximal end for manipulation by an operator and a distal end for operation within a patient. The distal end and proximal end preferably form one continuous piece, but need not be in a single piece. The delivery catheter 1312 may be used, for among other things, as a delivery system for delivering one or more radiofrequency electrodes to the desired site for nerve ablation. In an embodiment, the delivery catheter 1312 may have, for example, an outer diameter of about a 2.55 mm and about a 0.09 mm or less for an in inner diameter.

The device 1300 includes a guide wire 112 that may be advanced into the patient, e.g., through the delivery catheter 1312. The guide wire 112, extends, through (within) the delivery catheter 1312. The guide wire 112 here has a 0.035″ thickness (or could employ other thicknesses as known in the art). The guide wire 112 is inserted into the patient's vascular system, e.g. through the groin, and advanced to the desired location. Next, the delivery catheter 1312 is inserted into the patient and threaded over the guide wire 112 to the desired location. The device 1300 may be advanced to the desired location within the patient's vascular system with, e.g., a rapid exchange (RX) or over-the-wire wire (OTW) delivery system, with the 0.035″ or smaller guide wire 112 employed for the device 1300. Radiographic control may be employed and contrast media may also be injected at the beginning of the procedure to assist in manipulation and positioning of the instruments.

The device 1300 includes one or more electrodes 1316 deployable from the delivery catheter 1312 adjacent the distal end of the delivery catheter 1312. At least a single electrode is used. The one or more electrodes 1316 are capable of conducting radiofrequency (RF) energy. Initially, or when the one or more electrodes 1316 are in a non-deployed position, the one or more electrodes 1316 are located within the delivery catheter 1312. The one or more electrodes 1316, when deployed, from a ring-shape structure generally positioned in a circular configuration centered around the delivery catheter 1312, such that the one or more electrodes 1316 provide essentially 360° coverage at the target neurovascular region, for example, a renal artery ostium.

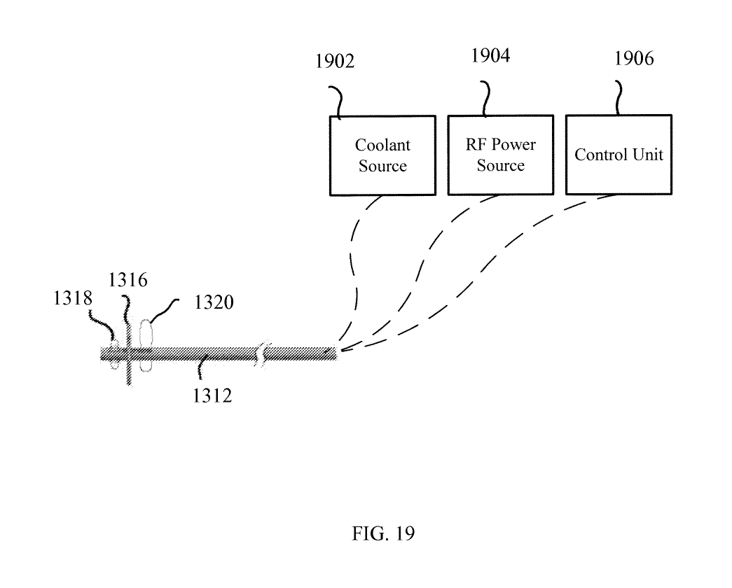

The electrode 1316 may be in the form of a hollow tube, for example, a nitinol or other nickel-titanium alloy hypotube. The hollow tube 1316 may be connected to a coolant source (e.g., coolant source 1902 illustrated in FIG. 19), for example, a cold saline solution, and other coolants. The coolant may be circulated through the hollow tube, when performing the ablative function. The coolant may also be discharged into the patient through an end of the hypotube, and the coolant may be carried out of the patient through the patient's blood stream. The use of the coolant may assist in controlling the ablative temperature of or applied to the tissue to be ablated, and reduce thermal injury to the aorta and renal artery, in particular the intima of the vessels. By cooling the tissue directly near the electrode, a target region deeper in the tissue (for example, tissue deep behind the ostium) can be ablated without ablating the tissue in direct contact with the electrode. This allows the target nerve region, a region wrapped around the outside of the aorta and the renal arteries, to be ablated when the device is deployed within the aorta and renal arteries. Thus, the device can be used for interaortic renal artery ablation for renal artery sympathetic neural ablation.

The nickel-titanium alloy or nitinol hypotube of the electrode 1316 is an alloy that has both super-elasticity and shape memory, i.e., remembers its original cold-forged shape, and returns to a pre-deformed shape when heated. This allows the electrode 1316 to be deformed during the retracting and deploying of the electrode 1316 into and from the delivery catheter 1312, and when heated, for example, by application of radiofrequency (RF) energy, form the ring-shape structure described herein.

As illustrated in FIG. 13, there is one electrode 1316. The electrode 1316 includes a stem portion extending from an aperture (for example, an electrode aperture 1422 described below), and a curved portion extending from the stem portion, forming ring-shape structure or arc around the delivery catheter 1312. When more than one electrode 16 is used (for example, as described below with reference to FIG. 6), the electrodes 1316 may be positioned such that, when the device 1300 is in a deployed position, the electrodes 1316 together form the ring-shape structure or are oriented concentrically, such that they together provide essentially 360° coverage around a target area. When electrode 1316 is used, the more than one electrodes 1316 may be nested or placed in parallel so as to form the ring-shape structure. Each of the nested electrodes 1316 may include a stem portion, and a first portion curving or extending from the stem portion. The plural electrodes are spaced around the axis of the catheter so that the curved portions form a rough circular shape when deployed.

The one or more electrodes 1316 may include a braid, coil, or laser cut tubular covering over the one or more electrodes 1316. This tubular covering may be used in the deployment and retraction of the one or more electrodes 1316 from the delivery catheter 1312. The tubular covering may also function to adjust a diameter of the ring-shape structure to deploy the one or more electrodes 1316 such that the ring-shape structure provides essentially 360° coverage around a target area.

The device 1300 may include one or more positioning elements 1318 deployable from the delivery catheter 1312 adjacent the distal end of the delivery catheter 1312. Initially, or when the one or more positioning elements 1318 are in a non-deployed position, the one or more positioning elements 1318 are located within the delivery catheter 1312. The one or more positioning elements 1318 are deployable from the delivery catheter 1312 at a target region from a position of the delivery catheter 1312 further distal than the one or more electrodes 1316. In use, this allows the positioning elements 1318 to position and secure device 1300 at the desired location within a vessel, e.g., the aorta in the area of the renal artery ostium. The one or more positioning elements 1318 may be used so that the one or more electrodes 1316 may operate at the precise location, namely around the renal artery ostium. Otherwise, if the device 1300 is not properly positioned, the electrode(s) 1316 could ablate tissue that is not intended to be affected, causing undesired damage. If the RF electrode 1316 is circularly configured, the positioning elements 1318 may center the electrode 1316 circumferentially around the renal artery ostium, namely the opening to the renal artery.

The one or more positioning elements 1318 may be wire loops and are located symmetrically around the delivery catheter 1312. When the delivery catheter 1312 is inserted at least partially into the entrance of the renal artery, the positioning elements 1318 may be deployed approximately to the diameter of the renal artery, so as to locate the electrode 1316 from being moved distally or proximally relative to the renal artery, so as to allow the device 1300 to hold its position within the renal artery relative to the aorta. When the device 1300 is so positioned by the positioning elements 1318, the electrode 1316 may then be positioned against the renal artery ostium to perform the ablative function, as will be shortly described.

The device 1300 may also include one or more pressing elements 1320 deployable from the delivery catheter 1312 proximal to the electrode 1316. Initially, or when the one or more pressing elements 1320 are in a non-deployed position, the one or more pressing elements 1320 are located within the delivery catheter 1312. The one or more pressing elements 1320 are deployable from the delivery catheter 1312 once the device is at a target nerve region, and from a position of the delivery catheter 1312 more proximal than the one or more electrodes 1316. When deployed, the pressing elements 1320 may be used to press the deployed one or more electrodes 1316 against the tissue to be ablated.

The pressing elements 1320 may be wire loops and may be located symmetrically around the delivery catheter 1312. The pressing elements 1320 may advance the delivery catheter 1312 distally such that the delivery catheter 1312 presses against a proximally-facing surface of the one or more electrodes 1316 to then be manipulated to push the one or more electrodes 1316 distally against the renal artery ostium. When the one or more electrodes 1316 are pressed up against the renal artery ostium of the aorta, the one or more electrodes 1316, which are positioned in a circular configuration, contact the renal artery ostium of the aorta. Heat may then be generated to the one or more electrodes 1316 by supplying a suitable RF energy source, and the ablation is performed for the elimination (or interruption) of nerve activity, such as nerve activity that leads specifically to the kidney.

Each of the one or more electrodes 1316, the one or more positioning elements 1318 and the one or more pressing elements 1320 may be selectively and independently movable between a non-deployed position (or retracted) and a deployed position, and back to the non-deployed position. Alternatively, they could be joined in a manner such that they are deployed together as a group (e.g., all of the positioning elements 1318 are deployed together). In the non-deployed position, as illustrated in FIG. 14 the electrode 1316, the positioning elements 1318 and the pressing elements 1320 of device 1300 are retracted within the delivery catheter 1312. As illustrated in FIG. 14, the delivery catheter 1312 includes an electrode aperture 1422, positioning element apertures 1424 and pressing element apertures 1426. The electrode aperture 1422, the positioning element apertures 1424 and the pressing element apertures 1426 allow the electrode 1316, the positioning elements 1318 and the pressing elements 1320 to be extended out of the delivery catheter 1312, to their respective deployed positions.

In an embodiment, a distance between the distal end of the delivery catheter 1312 and the electrode aperture 1422 and/or the electrode 1316 is about 10 mm to about 20 mm in length. A distance between the positioning element apertures 1424 and the electrode aperture 1422 and/or the electrode 1316 is about 5 mm to about 7 mm in length.

In the non-deployed position, the delivery catheter 1312 is advanced longitudinally through the blood vessel, e.g., over guide wire 112, to the relevant location within the body lumen, such as within the aorta, and into the desired position within the inner circumference of the vessel, such as at the renal artery ostium of the aorta. Once at the desired position, the electrode 1316, the positioning elements 1318 and the pressing elements 1320 are deployed. Preferably, the positioning elements 1318 are deployed first, then the electrode 1316 followed by the pressing elements 1320. This order need not be the only order, however.

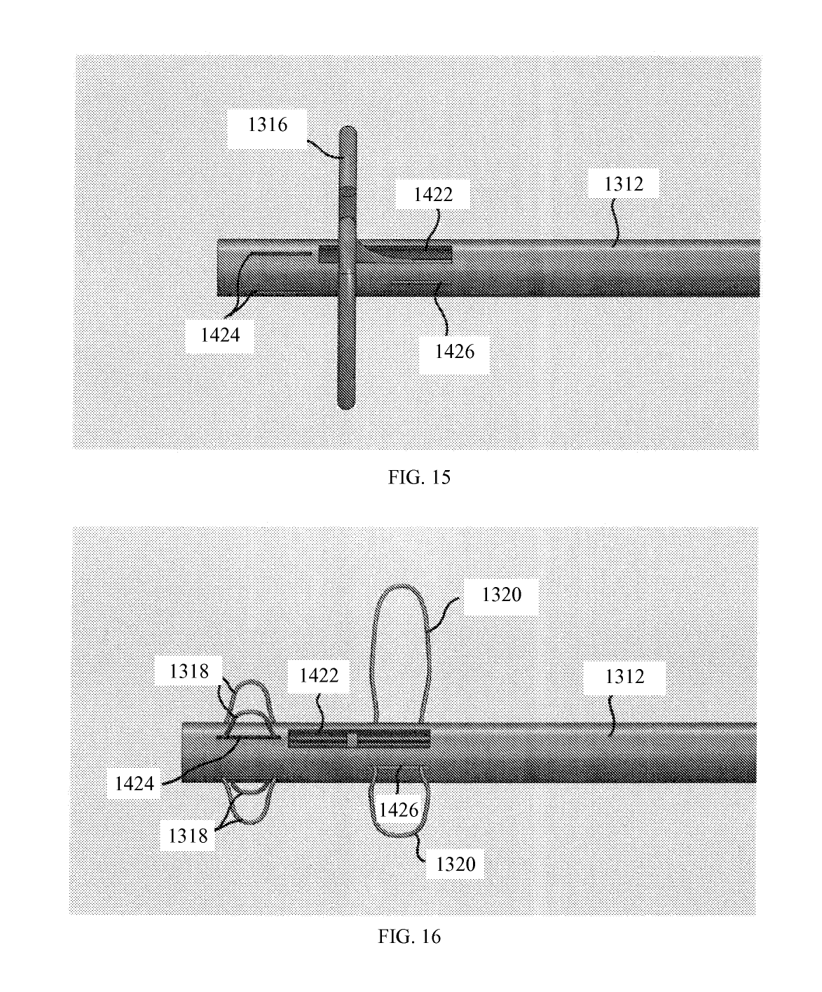

As illustrated in FIG. 15, the electrode 1316 is in the deployed position for operation within the patient. In the deployed position, the electrode 1316 extends out of the delivery catheter 1312 through the electrode aperture 1422, forming the ring-shape structure generally positioned in a circular configuration centered around the delivery catheter 1312, such that the electrode 1316 provides essentially 360° coverage at the target nerve region. The electrode 1316 can be pressed up against and put into contact with the renal artery ostium of the aorta, for instance, to ablate the nerve activity circumferentially around the ostium.

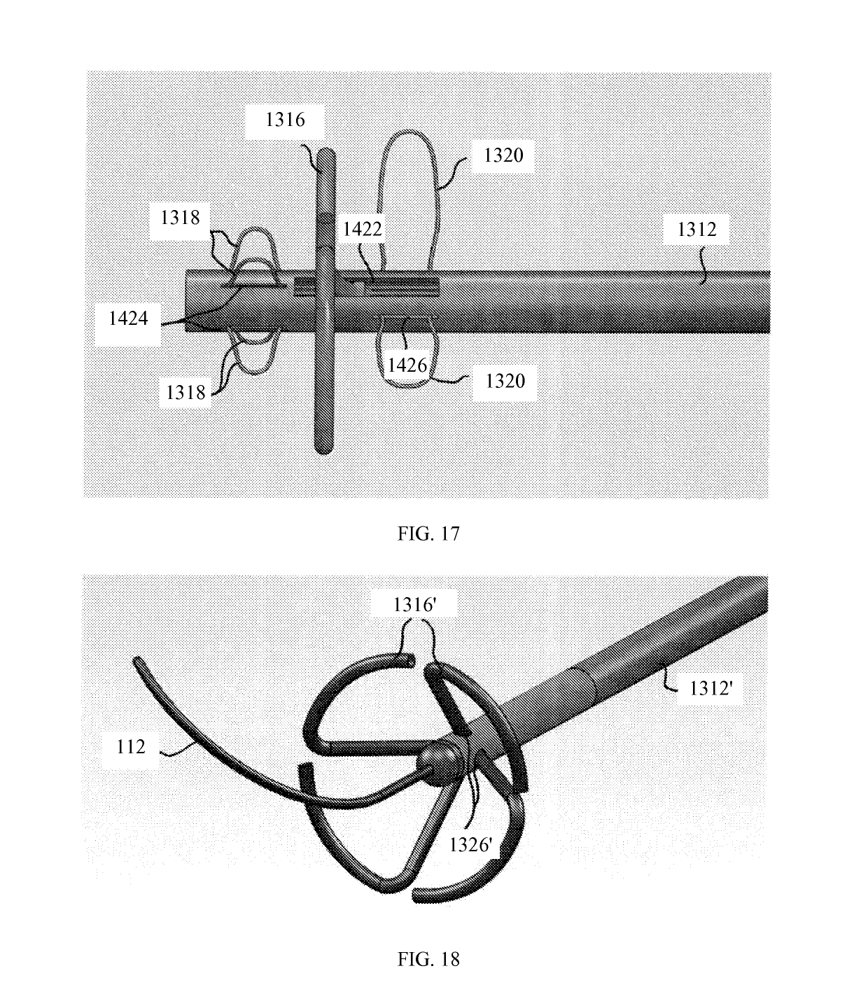

As illustrated in FIGS. 16 and 17, the positioning elements 1318 and the pressing elements 1320 are in the deployed position, for operation within the patient. In the deployed position, the positioning elements 1318 and the pressing elements 1320 extend out of the delivery catheter 1312 through the positioning element apertures 1424 and the pressing element apertures 1426, respectively. Referring to FIG. 17, the electrode 1316, the positioning elements 1318 and the pressing elements 1320 are all in the deployed position for operation within the patient.

To return to the non-deployed position, as for withdrawal, the electrode 1316, the positioning elements 1318 and the pressing elements 1320 are retracted into the inner diameter of delivery catheter 1312.

In another embodiment, as illustrated in FIG. 18, the device includes more than one electrodes 1316′ deployable from a delivery catheter 1312′ adjacent the distal end of the delivery catheter 1312′. The electrodes 1316′ are similarly capable of conducting RF energy. Initially, or when the electrodes 1316′ are in a non-deployed position, the electrodes 1316′ are located within the delivery catheter 1312′. The electrodes 1316′, when deployed, are positioned such that, when the device is in a deployed position, the electrodes 1316′ together form a ring-shape structure, or are oriented concentrically, such that they together provide (perhaps roughly) essentially 360° coverage around a target area. As illustrated in FIG. 18, there are four electrodes 1316′, but there can be fewer electrodes or more electrodes, each of which include a stem portion extending radially from the respective aperture 1426′ in the delivery catheter 1312′, and a curved portion extending from the stem portion. The curved portions align to form a ring-shape structure or arc around the delivery catheter 1312′. The electrodes 1316′ may also include the braid, coil, or laser cut tubular covering over the electrodes 1316′, as described above with reference to electrode 1316.

The delivery catheter 1312′ also includes electrode apertures 1426′ to allow the electrodes 1316′ to be extended out of the delivery catheter 1312′ to their respective deployed positions. Although not shown, the device may also include the one or more positioning elements, the one or more pressing elements, and the delivery catheter 1312′ may include their respective apertures, such that the device functions is essentially the same manner as described above with respect to FIGS. 13-17.

In these embodiments, the positioning elements 1318 operate to position, center, and secure the device at the desired location. This is accomplished by insertion of the unexpanded distal end of the delivery catheter 1312/1312′ at least partially into the entrance of the renal artery so as to serve, by deployment of the positioning elements 1318, as an anchor for the device within the aorta so that the electrodes 1316/1316′ can perform their ablative function. Similarly, the one or more pressing elements 1320 operate to engage the one or more electrodes 1316/1316′ at the desired location. This is accomplished by using the pressing elements 1320 so as to push the one or more electrodes 1316/1316′ against the tissue to be ablated so that the one or more electrodes 1316/1316′ can perform their ablative function.

The proximal end of the device may include at least one port for connection to a source of radiofrequency (RF) power (e.g., RF power source 1904 illustrated in FIG. 19). The device can be coupled to a source of RF energy, such as RF in about the 300 kilohertz to 500 kilohertz range. The electrodes 1316/1316′ may be electrically coupled to the RF energy source through this port. The device may also be connected to coolant source, and a control unit for sensing and measurement of other factors, such as temperature, conductivity, pressure, impedance and other variables, such as nerve energy.

The one or more electrodes 1316/1316′ may be electrically connected to the radiofrequency (RF) energy source. The RF energy source may be an external RF control unit that provides RF energy to the one or more electrodes 1316/1316′. All the electrodes 1316/1316′ may be attached to the same wire such that they are made to operate together, or the electrodes 1316/1316′ may have wires that loosely connect them, in order for them to be connected electrically.

There may also be multiple wires, each of which is attached to one or more of the electrodes 1316/1316′ so as to conduct RF energy from the RF control unit to individual electrodes 1316/1316′. This allows independent control of the electrodes 1316/1316′ to deliver RF energy simultaneously or in a sequential or other desired pattern.

The one or more electrodes 1316/1316′ operate to provide radiofrequency energy for heating of the desired location during the nerve ablation procedure. The one or more electrodes 1316/1316′ may be constructed of any suitable conductive material, as is known in the art. Examples include stainless steel and platinum alloys.

As described above, the one or more electrodes 1316/1316′ are in a preferred form, hollow tubes, for example, nitinol hypotubes. An example of a nitinol hypotubes may be a 4×0.018 mm nitinol hypotube. The hollow tube may be connected to a coolant source (e.g., coolant source 1902 illustrated in FIG. 19), for example, a cold saline solution, and other coolants both gas and liquid. The coolant is circulated through the hollow tube, when performing the ablative function. This may assist in controlling the ablative temperature applied to the tissue to be ablated, and reduce thermal injury to the aorta and renal artery. For example, this may limit the thermal effect to about a 3 mm to about a 6 mm depth, for example, from the level of the renal artery ostium.

The cooling allows a target region deeper in the tissue (for example, tissue deep behind the ostium) to be ablated without ablating the tissue in close proximity to the electrode. This allows the target nerve region, a region wrapped around the outside of the aorta and the renal arteries, to be ablated.

The one or more electrodes 1316/1316′ may operate in either bipolar or monopolar mode, with a ground pad electrode. In a monopolar mode of delivering RF energy, a single electrode is used in combination with an electrode patch that is applied to the body to form the other electrical contact and complete an electrical circuit. A bipolar operation is possible when two or more electrodes are used, such as two concentric electrodes. The one or more electrodes 1316/1316′ may be attached to an electrode delivery member, such as the wire frame, by the use of soldering or welding methods which are well known to those skilled in the art.

The one or more electrodes 1316/1316′ are oriented in a generally circular configuration. The diameter of the circular or ring-shape of the electrodes 1316/1316′ is determined by the width of the aortic artery branch for which denervation is desired. If the diameter of the circular or ring-shape of the electrodes 1316/1316′ is smaller than the diameter of the aortic artery branch for which denervation is desired, the one or more electrodes 1316/1316′ would not actually be in contact with tissue, and no ablation would occur. For example, when aortic denervation is desired at the level of the renal artery ostium, which is approximately 6-7 mm in diameter at the ostium of the aorta, the diameter of the circular or ring-shape of the electrodes 1316/1316′ should be at least that distance, i.e., 7 mm, in order to properly provide ablation surrounding the renal artery ostium. The diameter of the circular or ring-shape of the electrodes 1316/1316′ may be calculated with reference to the renal artery ostium. For example, if it is desired that the RF energy be applied at least approximately 2 mm from each edge of the renal artery ostium, the diameter of the circular or ring-shape of the electrodes 1316/1316′ that surround the imaging catheter may have a 10 mm to about a 15 mm diameter.

The one or more electrodes 1316/1316′ can be disposed to treat tissue by delivering radiofrequency (RF) energy. The radiofrequency energy delivered to the electrode may have a frequency of about 5 kilohertz (kHz) to about 1 GHz. In specific embodiments, the RF energy may have a frequency of about 10 kHz to about 1000 MHz; specifically about 10 kHz to about 10 MHz; more specifically about 50 kHz to about 1 MHz; even more specifically about 300 kHz to about 500 kHz.

Each electrode may be operated separately or in combination with another as sequences of electrodes disposed in arrays. Treatment can be directed at a single area or several different areas of a vessel by operation of selective electrodes. An electrode selection and control switch may include an element that is disposed to select and activate individual electrodes.

The RF power source may have multiple channels, delivering separately modulated power to each electrode. This reduces preferential heating that occurs when more energy is delivered to a zone of greater conductivity and less heating occurs around electrodes that are placed into less conductive tissue. If the level of tissue hydration or the blood infusion rate in the tissue is uniform, a single channel RF power source may be used to provide power for generation of lesions relatively uniform in size.

The RF energy delivered through the electrodes to the tissue causes heating of the tissue due to absorption of the RF energy by the tissue and ohmic heating due to electrical resistance of the tissue. This heating can cause injury to the affected cells and can be substantial enough to cause cell death, a phenomenon also known as cell necrosis. For ease of discussion, “cell injury” includes all cellular effects resulting from the delivery of energy from the electrodes up to, and including, cell necrosis. Use of the catheter device can be accomplished as a relatively simple medical procedure with local anesthesia. In an embodiment, cell injury proceeds to a depth of approximately 1-5 mm from the surface of the mucosal layer of sphincter or that of an adjoining anatomical structure.

Also to be potentially included in this design is a means to measure renal nerve afferent activity prior to and following RF nerve ablation. By measuring renal nerve activity post procedure, a degree of certainty is provided that proper nerve ablation has been accomplished. Renal nerve activity may be measured through the same mechanism as that required for energy delivery and the electrodes.

Nerve activity may be typically measured by one of two means. Proximal nerve stimulation can occur by means of transmitting an electrical impulse to the catheter. Action potentials can be measured from the segment of the catheter situated within a more distal portion of the nerve. The quantity of downstream electrical activity as well as the time delay of electrical activity from the proximal to distal electrodes will be provide a measure of residual nerve activity post nerve ablation. The second means of measuring nerve activity is to measure ambient electrical impulses prior to and post nerve ablation within a site more distal than the ablation site.

The one or more electrodes 1316/1316′ may operate to provide radiofrequency energy for both heating and temperature sensing. Thus, the one or more electrodes 1316/1316′ can be used for heating during the ablation procedure and can also be used for sensing of nerve activity prior to ablation as well as after ablation has been done.

The one or more electrodes 1316/1316′ may also be coupled to a sensor or a control unit (e.g., control unit 1906 illustrated in FIG. 19) capable of measuring such factors as temperature, conductivity, pressure, impedance and other variables. For example, the device may have a thermistor that measures temperature in the lumen, and a thermistor may be a component of a microprocessor-controlled system that receives temperature information from the thermistor and adjusts wattage, frequency, duration of energy delivery, or total energy delivered to the one or more electrodes 1316/1316′. In other words, a closed loop, feedback control system may be incorporated to optimize the delivery of ablative energy to the tissue.

The device may also be coupled to a visualization apparatus, such as a fiber optic device, a fluoroscopic device, an anoscope, a laparoscope, an endoscope or the like. In an embodiment, devices coupled to the visualization apparatus are controlled from a location outside the body, such as by an instrument in an operating room or an external device for manipulating the inserted catheter.

The device may be constructed with markers that assist the operator in obtaining a desired placement, such as radio-opaque markers, etchings or microgrooves. Thus, device may be constructed to enhance its imageability by techniques such as ultrasounds, CAT scan or MRI. In addition, radiographic contrast material may be injected through a hollow interior of the catheter through an injection port, thereby enabling localization by fluoroscopy or angiography.

The disclosure herein also comprises a method for ablation of renal artery nerve function within the aorta using the devices described herein. A method for performing ablation of a nerve at an artery ostium includes inserting a distal end of a device, for example, device 1300 including the delivery catheter 1312/1312′, at a target nerve region using a guide wire. The targeted neurovascular region may be the renal artery ostium.

This method includes deploying one or more positioning elements, for example, positioning elements 1318, from the delivery catheter 1312/1312′ to position the device and an electrode, for example, electrodes 1316/1316′, for deployment within the target nerve region. As described above, the positioning elements may center and secure the device, for example, the delivery catheter 1312/1312′, in the target nerve region.

The method includes deploying the electrode, for example, electrodes 13161316′, from the delivery catheter 1312/1312′ at the target nerve region. When deployed, the electrode may form a ring-shaped structure generally centered around the delivery catheter 1312/1312′ adjacent the distal end. The ring-shaped structure may also extend substantially circumferentially around the target nerve region.

The method of this embodiment includes deploying one or more pressing elements, for example, pressing elements 1320, from the delivery catheter 1312/1312′ (either before or after electrode deployment) at a position more proximal than the electrode, for example, electrodes 1316/1316′. As described above, the pressing elements may be used for pressing the deployed electrode, for example, electrodes 1316/1316′, against tissue to be ablated at the target nerve region. In an embodiment, the method may also include pressing the deployed electrode, for example, electrodes 1316/1316′, against tissue at the target nerve region.

Radiofrequency (RF) energy is applied through the deployed electrode, for example, electrodes 1316/1316′, in an amount to ablate tissue at the target nerve region. The radiofrequency energy may be applied at a single energy level for a defined and regulated period of time or at a first energy level and at least a second energy level which is different from the first energy level. The first and second energy levels may be alternated and pulsed. Further, there may be a defined pause between the delivery of each energy level to allow the tissue temperature to normalize.

The method may include circulating a coolant through the hollow tube electrodes during the ablation procedure.

The method may include a step of precooling the target nerve area, for example by circulating the coolant through the hollow tube electrodes. The precooling may be performed for any period of time, particularly about 10 seconds to about 20 seconds, and more particularly for about 15 seconds. Following the precooling step, the radiofrequency energy may be applied at the first energy level. The first energy level is about 1.4 amps, and is applied for about 60 seconds to about 90 seconds. Following the application of the radiofrequency energy at the first energy level, the radiofrequency energy may be applied at the second energy level. The second energy level is about 1.2 amps, and is applied for about 90 seconds. A pause may also be incorporated between the delivery of the first and second energy level.

The ablation procedure may include applying the radiofrequency energy at a first energy level for a first period of time, followed by a rest and then applying the radiofrequency energy at a second energy level for a second period of time. The first energy level and the second energy level may be equal. Similarly, the first period of time and the second period of time may be equal.

Although the method steps are described herein serially, there is no particular requirement that the method be performed in the same order in which this description lists the steps, except where so indicated.

Although the devices, systems, and methods have been described and illustrated in connection with certain embodiments, many variations and modifications will be evident to those skilled in the art and may be made without departing from the spirit and scope of the disclosure. The discourse is thus not to be limited to the precise details of methodology or construction set forth above as such variations and modification are intended to be included within the scope of the disclosure.

What is claimed is:

1. An ablation device, as for sympathetic aortic and renal artery denervation, comprising:
a catheter delivery mechanism including an elongated tube with a distal end and a proximal end, said distal end being emplaceable within an arterial system for delivery within an aorta at a level of a renal artery ostium; and
at least one radiofrequency electrode initially located within said tube, said electrode being deployable from said tube, said electrode when deployed forming a ring-shaped structure generally centered about said tube adjacent said distal tube end.

2. The ablation device of claim 1, further including at least one positioning element initially located within said tube, said at least one positioning element being deployable from said tube from a position of said tube further distal than said electrode.

3. The ablation device of claim 1, further including at least one pressing element initially located within said tube, said at least one pressing element being deployable from said tube more proximal than said electrode for use in pressing said deployed electrode against tissue to be ablated.

4. The ablation device of claim 1, further including a source of radiofrequency energy connected to said electrode.

5. The ablation device of claim 1, wherein said electrode is a hollow tube.

6. The ablation device of claim 5, further including a source of coolant, wherein said coolant is circulated through said electrode tube.

7. The ablation device of claim 1, wherein said electrode is comprised of a plurality of separate electrode members each of which is deployable from said tube, and together take a ring-like shape when deployed.

8. The ablation device of claim 7, wherein said electrode members are in the form of hollow tubes, further including a source of coolant, wherein said coolant is circulated through said electrode tube members.

9. The ablation device of claim 4, wherein said radiofrequency energy is applied at at least two different energy levels.

10. The ablation device of claim 2, wherein said positioning elements are wire loops.

11. The ablation device of claim 10, wherein said wire loops are located symmetrically about said tube.

12. The ablation device of claim 3, wherein said pressing elements are wire loops.

13. The ablation device of claim 12, wherein said wire loops are located symmetrically about said tube.

14. The ablation device of claim 7, wherein said electrode members when deployed have a stem portion extending generally radially from a respective port in said tube, and a curved portion extending from said stem in an arc about said tube.

15. A method for performing ablation of a nerve at an artery ostium, as for denervation, comprising:
providing a catheter delivery mechanism including an elongated tube with a distal end and a proximal end, said distal end being emplaceable within a body lumen at a target nerve region, and having a guide wire within said elongated tube;
inserting said catheter delivery mechanism within an arterial system with the distal end at said renal artery ostium using said guide wire;
providing at least one radiofrequency electrode initially located within said tube, said electrode when deployed forming a ring-shaped structure generally centered about said tube adjacent said distal tube end;
deploying said electrode at said renal artery ostium; and
applying radiofrequency energy through said deployed electrode from said tube at said renal artery ostium in an amount to ablate tissue around said renal artery ostium.

16. The ablation method of claim 15, further including deploying one or more positioning elements initially located within said tube from a position of said tube further distal than said electrode to position said electrode.

17. The ablation method of claim 15, further including deploying one or more pressing elements initially located within said tube from a position more proximal than said electrode for use in pressing said deployed electrode against tissue as said target nerve region.

18. The ablation method of claim 15, wherein said electrode is a hollow tube.

19. The ablation method of claim 18, further including a source of coolant, and circulating said coolant through said electrode tube during ablation.

20. The ablation method of claim 15, wherein said electrode is comprised of a plurality of separate electrode members each of which is deployable from said tube, and together take a ring-like shape when deployed.

21. The ablation method of claim 20, wherein said electrode members are in the form of hollow tubes, further including a source of coolant, and circulating said coolant through said electrode tube member during ablation.

22. The ablation method of claim 15, wherein said radiofrequency energy is applied at a first energy level and at least a second energy level which is different from said first energy level.

23. The ablation method of claim 22, wherein said first and second energy levels are alternated and pulsed.

24. A method for performing ablation of a renal nerve at the renal artery ostium, comprising:
providing a catheter delivery mechanism including an elongated tube with a distal end and a proximal end, said distal end being emplaceable within the body lumen at the renal artery ostium, and having a guide wire within said elongated tube for positioning said catheter delivery mechanism;
inserting said catheter delivery mechanism with its distal end at the renal ostium;
providing at least one radiofrequency electrode initially located within said tube, said electrode when deployed forming a ring-shaped structure generally centered about said tube adjacent said distal tube end;
providing a plurality of positioning elements initially located within said tube, said positioning elements being deployable from said tube in the renal artery at the ostium from a position of said tube further distal than said electrode;
deploying said positioning elements to position said electrode;
deploying said electrode;
providing a plurality of pressing elements initially located within said tube, said pressing elements being deployable from said tube more proximal than said electrode for use in pressing said deployed electrode against ostium tissue to be ablated;
pressing said electrode against the ostium tissue; and
applying radiofrequency energy through said deployed electrode from said tube in an amount to ablate the ostium tissue.

25. The ablation method of claim 24, wherein the method is used to treat hypertension.

26. The ablation method of claim 25, wherein said electrode is a hollow tube.

27. The ablation method of claim 25, further including a source of coolant, and circulating said coolant through said electrode tube during ablation.

28. The ablation method of claim 25, wherein said electrode is comprised of a plurality of separate electrode members each of which is deployable from said tube, and together take a ring-like shape when deployed.

29. The ablation method of claim 28, wherein said electrode members are in the form of hollow tubes, further including a source of coolant, and circulating said coolant through said electrode tube member during ablation.

30. The ablation method of claim 29, wherein said radiofrequency energy is applied at a first energy level and at least a second energy level which is different from said first energy level.

31. A nerve-ablation device, comprising:
a catheter delivery apparatus having a portion which yields a cylindrical shape having a longitudinal axis in use;
at least two radiofrequency delivery electrodes positioned in a helical configuration around an outer surface of said cylindrical shape; and
electrical connections for said electrodes to connect to a radiofrequency source.

32. The nerve-ablation device of claim 31, wherein said cylindrical shape is a balloon portion of said catheter delivery apparatus.

33. The nerve-ablation device of claim 31, wherein said cylindrical shape is a expansible wire frame portion of said catheter delivery apparatus.

34. The nerve-ablation device of claim 32, wherein said balloon portion is separated from said radiofrequency electrodes by an insulation pad that protects said balloon portion from high temperature.

35. The nerve-ablation device of claim 31, further including a mechanism using said radiofrequency electrodes to also monitor one or more physical conditions at a site of use.

36. A nerve-ablation device, comprising:
a catheter delivery apparatus having a portion which yields an expanded shape having a central axis in use, and presents a broadened distal face surface in said expanded shape;
at least one radiofrequency delivery electrode positioned on an outer surface of said distal face and generally concentric with said central axis; and
a mechanism associated with said expanded shape for pressing said distal face surface against tissue to be ablated.

37. The nerve-ablation device of claim 36, wherein said radiofrequency electrode assumes a circular configuration in use.

38. The nerve-ablation device of claim 36, wherein said portion which yields an expanded shape is a balloon.

39. The nerve-ablation device of claim 36, further including a positioning mechanism located forwardly of said distal face, said positioning mechanism being sized to be received in a body lumen.

40. The nerve-ablation device of claim 39, wherein said positioning mechanism is an inflatable member.

40. The nerve-ablation device of claim 36, wherein said portion which yields an expanded shape is a wire frame.

41. The nerve-ablation device of claim 36, wherein said catheter delivery apparatus comprises an elongated sheath, said sheath having a lateral port, said portion which yields an expanded shape being initially collapsed and movable within said sheath, and further including a delivery member which moves said portion which yields an expanded shape within said sheath through said port and into a deployed position.
